(19) United States
(12) Patent Application Publication
(10) Pub. No.: US 2022/0380838 A1
(43) Pub. Date: Dec. 1, 2022

(54) METHODS AND COMPOSITIONS FOR ANALYTE DETECTION AND PROBE RESOLUTION

(71) Applicant: 10x Genomics, Inc., Pleasanton, CA (US)

(72) Inventors: Malte KÜHNEMUND, Stockholm (SE); Xiaoyan QIAN, Stockholm (SE)

(21) Appl. No.: 17/829,072

(22) Filed: May 31, 2022

Related U.S. Application Data
(60) Provisional application No. 63/195,613, filed on Jun. 1, 2021.

(51) Int. Cl. C12Q 1/6841; C12Q 1/6818; C12Q 1/682; C12Q 1/6874

(52) U.S. Cl. CPC C12Q 1/6841; C12Q 1/6818; C12Q 1/682; C12Q 1/6874

(57) ABSTRACT

The present disclosure in some aspects relates to methods and compositions for accurately detecting and quantifying analytes present at high levels, such as highly expressed genes in a sample. In some embodiments, a probe-resolution barcode sequence disclosed herein does not specifically correspond to any particular target analyte(s) but can be used to resolve dense optical signals due to spatially overlapping signals associated with different molecules of a target analyte, thereby enabling resolution of signals in a dense “spot” and accurate counting of spots associated with molecules that are in spatial proximity. Also provided are kits comprising probes for use in such methods.

### CROSS-REFERENCE TO RELATED APPLICATION

This application claims priority to U.S. Provisional Patent Application No. 63/195,613, filed Jun. 1, 2021, entitled “METHODS AND COMPOSITIONS FOR ANALYTE DETECTION AND PROBE RESOLUTION,” which is herein incorporated by reference in its entirety for all purposes.

### FIELD

The present disclosure generally relates to methods and compositions for detecting a plurality of molecules of one or more analytes in a sample.

### BACKGROUND

In multiplex assays where multiple signals are detected simultaneously, it is important that each individual signal can be distinguished from one another so that as much information as possible can be collected from the assays. For example, in microscopy-based optical assays, individual “spots” emitting optical signals often need to be resolved from adjacent spots in a sample. However, resolving a large number of signals of varying strengths remains challenging, and improved methods are needed. The present disclosure addresses these and other needs.

### BRIEF SUMMARY

During in situ analysis such as those involving sequencing-by-hybridization (SBH), highly expressed analytes can produce many locally amplified probes in close proximity, causing optical crowding and limiting the dynamic range for quantification. Large signal spots (e.g., due to high analyte abundance) may overlap with one another and/or mask adjacent smaller signal spots, rendering the smaller spots unresolvable. In addition, when bright spots and relatively dim spots are in the same microscope field of view, the dim spots may not pass the threshold of spot detection for subsequent image analysis. Thus, highly abundant analytes not only may render detection of the analytes themselves challenging, but also may lead to inability to detect nearby signal spots and/or weaker signal spots in the same field of view. As such, improved methods for precise detection and accurate quantification of the expression levels of highly expressed genes in a biological sample are needed. In some aspects, the present disclosure relates to methods and compositions for more accurately detecting and quantifying analytes present at high levels in a sample.

In some embodiments, disclosed herein is a method for analyzing a biological sample, comprising: (a) contacting the biological sample with a plurality of probes each comprising a target-specific barcode sequence, wherein a first probe of the plurality of probes comprises a first probe-resolution barcode sequence and a second probe of the plurality of probes comprises a second probe-resolution barcode sequence, wherein the first probe targets a first molecule of a target analyte and the second probe targets a second molecule of the target analyte in the biological sample, and the target-specific barcode sequence corresponds to the target analyte; (b) detecting a plurality of signals associated with the target-specific barcode sequences of the plurality of probes; (c1) detecting a signal associated with the first probe-resolution barcode sequence; and (c2) detecting a signal associated with the second probe-resolution barcode sequence, wherein the signals of (c1) and (c2) are associated with detectable probes that hybridize to the first and second probe-resolution barcode sequences or complements thereof, and the first and second probe-resolution barcode sequences are detected separately (e.g., not detected at the same time). In some embodiments, the method further comprises (d) resolving the plurality of signals indicative of the target analyte detected in step (b) using signals detected in steps (c1) and (c2) and attributed to the first and second probes, respectively.

In some embodiments, disclosed herein is a method for analyzing a biological sample, comprising contacting the biological sample with a plurality of probes each comprising a target-specific barcode sequence, wherein a first probe of the plurality of probes comprises a first probe-resolution barcode sequence and a second probe of the plurality of probes comprises a second probe-resolution barcode sequence. In some embodiments, the plurality of probes target a target nucleic acid in the biological sample, and the target-specific barcode sequence corresponds to the target nucleic acid or a sequence thereof. In some embodiments, the first probe targets a first molecule of a target nucleic acid and the second probe targets a second molecule of the target nucleic acid in the biological sample, and the first and second molecules of the target nucleic acid can be at the same location or at different locations in the biological sample. In some embodiments, the first and second probe-resolution barcode sequences are distinct. In some embodiments, the first and second probe-resolution barcode sequences do not correspond to any particular target nucleic acid in the biological sample, but rather distinguish the first probe from the second probe, where both probes correspond to the same target nucleic acid.

In any of the embodiments disclosed herein, the method can further comprise detecting a plurality of signals associated with the target-specific barcode sequences of the plurality of probes. In any of the embodiments disclosed herein, the method can further comprise detecting a signal associated with the first probe-resolution barcode sequence. In any of the embodiments disclosed herein, the method can further comprise detecting a signal associated with the second probe-resolution barcode sequence. In some embodiments, each signal of the plurality of signals associated with the target-specific barcode sequences of the plurality of probes can be associated with the signal associated with the first probe-resolution barcode sequence or the signal associated with the second probe-resolution barcode sequence. For example, the signal associated with the target-specific barcode sequence (thus associated with the target analyte such as a target nucleic acid of interest) at a given location in the biological sample can be detected as a “spot.” The location of the “spot” can be registered and signals at that location in sequential probe hybridization and detection cycles can be tracked, associated and/or compared with signals from previous cycles, and/or compiled to generate a signal signature. Thus, the signal associated with the first or second probe-resolution barcode sequence can be associated with the target-specific barcode sequence (thus associated with the target analyte such as a target nucleic acid of interest). However, the signal associated with the first or second probe-resolution barcode sequence is only associated with a subset of the probes comprising the target-specific barcode sequence, and can be detected in a separate detection channel from other subset(s). As such, the signal associated with the first or second probe-resolution barcode sequence can be spatially resolved in cases where signals associated with the target-specific barcode sequence alone cannot be spatially resolved into individual puncta.

In any of the embodiments disclosed herein, the plurality of signals associated with the target-specific barcode sequences of the plurality of probes can comprise overlapping signals that are not spatially resolved into individual puncta. In any of the embodiments disclosed herein, for overlapping signals that are associated with the target-specific barcode sequence, each overlapping signal can be associated with the signal associated with the first probe-resolution barcode sequence or the signal associated with the second probe-resolution barcode sequence but not both, thereby resolving the overlapping signals associated with the target-specific barcode sequence into signals associated with the first and second probes, respectively.

In any of the embodiments disclosed herein, the plurality of signals associated with the target-specific barcode sequence can be detected at multiple locations in the biological sample, the signal associated with the first probe-resolution barcode sequence can be detected at a first subset of the multiple locations, the signal associated with the second probe-resolution barcode sequence can be detected at a second subset of the multiple locations, and the first and second subsets of the multiple locations do not completely overlap.

In any of the embodiments disclosed herein, the signals associated with the target-specific barcode sequence, the signal associated with the first probe-resolution barcode sequence, and/or the signal associated with the second probe-resolution barcode sequence can be detected using detectable probes that directly or indirectly bind to the target-specific barcode sequence or a complement thereof, the first probe-resolution barcode sequence or a complement thereof, and the second probe-resolution barcode sequence or a complement thereof, respectively, and optionally the detection can comprise rolling circle amplification (RCA), hybridization chain reaction (HCR), linear oligonucleotide hybridization chain reaction (LO-HCR), or primer exchange reaction (PER), or any combination thereof.

In any of the embodiments disclosed herein, the target-specific barcode sequence can be about 5, about 10, about 15, about 20, about 25, about 30, or about 35 nucleotides in length. In any of the embodiments disclosed herein, the first and second probe-resolution barcode sequences can be independently about 3, about 5, about 10, about 15, about 20, about 25, about 30, or about 35 nucleotides in length. In any of the embodiments disclosed herein, the target-specific barcode sequence can be about 20 nucleotides in length, and the first and second probe-resolution barcode sequences can be about 5 nucleotides in length.

In any of the embodiments disclosed herein, the first and/or second probes can further comprise an anchor sequence. In any of the embodiments disclosed herein, the anchor sequence can be adjacent to the target-specific barcode sequence, optionally wherein the anchor sequence can be separated from the 5′ or 3′ nucleotide of the target-specific barcode sequence by 0, 1, 2, 3, 4, 5, or more nucleotides. In any of the embodiments disclosed herein, the anchor sequence can be common between the first and second probes. In any of the embodiments disclosed herein, the anchor sequence can be common among the plurality of probes. In any of the embodiments disclosed herein, the anchor sequence can be common among probes targeting different target analytes in the biological sample. In any of the embodiments disclosed herein, the anchor sequence can be about 5, about 10, about 15, about 20, about 25, about 30, or about 35 nucleotides in length, optionally wherein the anchor sequence can be about 20 nucleotides in length.

In any of the embodiments disclosed herein, the first and/or second probes can further comprise one or more linker sequences. In any of the embodiments disclosed herein, the first and/or second probes can comprise two linker sequences flanking the first or second probe-resolution barcode sequence, respectively. In any of the embodiments disclosed herein, each of the one or more linker sequences can be independently 1, 2, 3, 4, 5, 6, 7, 8, 9 or more nucleotides in length. In any of the embodiments disclosed herein, the one or more linker sequences can be common between the first and second probes. In any of the embodiments disclosed herein, the one or more linker sequences can be common among the plurality of probes. In any of the embodiments disclosed herein, the one or more linker sequences can be common among probes targeting the same or different target analytes in the biological sample.

In any of the embodiments disclosed herein, the first and/or second probe-resolution barcode sequences can be adjacent to the target-specific barcode sequence, optionally wherein the first and/or second probe-resolution barcode sequences can be separated from the 5′ or 3′ nucleotide of the target-specific barcode sequence by 0, 1, 2, 3, 4, 5, or more nucleotides.

In any of the embodiments disclosed herein, the plurality of probes can further comprise a third probe comprising a third probe-resolution barcode sequence, and the method can further comprise detecting a signal associated with the third probe-resolution barcode sequence. In any of the embodiments disclosed herein, the plurality of probes can further comprise a fourth probe comprising a fourth probe-resolution barcode sequence, and the method can further comprise detecting a signal associated with the fourth probe-resolution barcode sequence. In any of the embodiments disclosed herein, the signals associated with the first, second, third, and/or fourth probe-resolution barcode sequences can be detected in separate detection channels, such as different fluorescent channels. As an example, detectable probes for the first, second, third, and fourth probe-resolution barcode sequences (or complements thereof) can be contacted with the biological sample all at once, and the signal associated with each probe-resolution barcode sequence can be detected in one of red, green, blue, and yellow fluorescent channels.

In any of the embodiments disclosed herein, the first, second, third, and/or fourth probe-resolution barcode sequences can be different among probes targeting the same target analyte (e.g., target nuclei acid).

In any of the embodiments disclosed herein, the first, second, third, and/or fourth probe-resolution barcode sequences can be common among two or more probes each targeting a different target analyte in the biological sample. In some instances, use of common probe-resolution barcode sequences minimizes the design burden of additional barcode sequences. In some instances, the common probe-resolution barcode sequences are an “add-on” feature of the probe design that provides additional resolution. For instance, a first pair of probes targeting Gene X and Gene Y respectively can share a common first probe-resolution barcode sequence, a second pair of probes targeting Gene X and Gene Y respectively can share a common second probe-resolution barcode sequence, a third pair of probes targeting Gene X and Gene Y respectively can share a common third probe-resolution barcode sequence, and a fourth pair of probes targeting Gene X and Gene Y respectively can share a common fourth probe-resolution barcode sequence.

In any of the embodiments disclosed herein, the first, second, third, and/or fourth probe resolution barcode sequences can be associated with the same species of organism. In any of the embodiments disclosed herein, the first, second, third, and/or fourth probe resolution barcode sequences can be associated with different species of organism. In some embodiments, the first molecule of the target analyte can be of a first species and the second molecule of the target analyst can be of a second species different from the first species, and the first and second probe-resolution barcode sequences can be associated with the first and second species, respectively.

In any of the embodiments disclosed herein, the target analyte can comprise a nucleic acid sequence, and the target analyte can optionally be a target DNA or RNA. In some embodiments, the plurality of probes can directly or indirectly bind to the same nucleic acid sequence in different molecules of the target analyte. In some embodiments, two or more of the plurality of probes each can directly or indirectly bind to a different nucleic acid sequence in different molecules of the target analyte.

In any of the embodiments disclosed herein, the first probe can comprise a first target binding sequence complementary to a first nucleic acid sequence of the target analyte and the second probe can comprise a second target binding sequence complementary to a second nucleic acid sequence of the target analyte. In some embodiments, the first and second target binding sequence can be the same. In some embodiments, the first and second target binding sequences can be different. In some embodiments, the first and second target binding sequences can hybridize to the same nucleic acid sequence in the target analyte. In some embodiments, the first and second target binding sequences can hybridize to different, adjacent, and/or partially overlapping nucleic acid sequences in the same nucleic acid molecule. For example, two or more probes can be designed with different target sequences that are tiled on the same nucleic acid molecule. In some instances, due to inefficiency of binding, one or some but not all probes that target the same nucleic acid molecule (e.g., targeting different sequences tiled on the nucleic acid molecule) binds to the nucleic acid molecule. In some embodiments, the adjacent nucleic acid sequences in the in the target analyte can be non-overlapping or partially overlapping. In some embodiments, the adjacent nucleic acid sequences in the target analyte can be separated by 0, about 5, about 10, about 15, about 20, or more nucleotides. In some embodiments, the adjacent nucleic acid sequences in the target analyte can be overlapping at about 2, about 5, about 10, about 15, about 20, or more nucleotides.

In any of the embodiments disclosed herein, the first and second probes can be circular probes or circularizable probes or probe sets. In any of the embodiments disclosed herein, the first and/or second probes can comprise a ribonucleotide, such as no more than four, no more than three, or no more than two ribonucleotides.

In any of the embodiments disclosed herein, the first and second probes can be circularized by ligation using a nucleic acid sequence in the target analyte and/or a splint as a template. In any of the embodiments disclosed herein, the first and second probes can be circularizable probes, and ends of the circularizable probes can be ligated using the nucleic acid sequence in the target analyte as a template, with or without gap filling prior to ligation. In any of the embodiments disclosed herein, the circularizable probes can comprise deoxyribonucleotides and/or ribonucleotide(s), and the target analyte can be DNA or RNA, optionally wherein the target analyte is a genomic DNA, an mRNA, a cDNA, or a reporter oligonucleotide (e.g., a report oligonucleotide directly or indirectly coupled to a binder such as an antibody). In any of the embodiments disclosed herein, the circularizable probe (e.g., a padlock probe) can comprise a 3′ ribonucleotide in a deoxyribonucleotide backbone.

In any of the embodiments disclosed herein, the ligation can comprise enzymatic ligation and/or chemical ligation, and/or the ligation can comprise template dependent ligation, and/or template independent ligation. In some embodiments, the enzymatic ligation can comprise using a ligase having an RNA-templated DNA ligase activity and/or an RNA-templated RNA ligase activity. In any of the embodiments disclosed herein, the enzymatic ligation can comprise using a ligase selected from the group consisting of a Chlorella virus DNA ligase (PBCV DNA ligase), a T4 RNA ligase, a T4 DNA ligase, and a single-stranded DNA (ssDNA) ligase. In any of the embodiments disclosed herein, the enzymatic ligation can comprise using a PBCV-1 DNA ligase or variant or derivative thereof and/or a T4 RNA ligase 2 (T4 Rn12) or variant or derivative thereof.

In any of the embodiments disclosed herein, the method can further comprise prior to the ligation, a step of removing molecules of the first probe, the second probe, and/or the splint that are not stably bound to the target analyte (e.g., target nucleic acid) from the biological sample, optionally the removing step can comprise one or more stringency washes.

In any of the embodiments disclosed herein, the method can further comprise generating products of the circularized first probe and the circularized second probe in situ in the biological sample. In any of the embodiments disclosed herein, the products can be amplification products generated using rolling circle amplification (RCA), optionally the RCA can comprise a linear RCA, a branched RCA, a dendritic RCA, or any combination thereof.

In any of the embodiments disclosed herein, the products can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In any of the embodiments disclosed herein, the products can be immobilized in the biological sample and/or crosslinked to one or more other molecules in the biological sample.

In any of the embodiments disclosed herein, the method can comprise imaging the biological sample to detect the products in situ by sequential hybridization, sequencing by hybridization, sequencing by ligation, sequencing by synthesis, sequencing by binding, or a combination thereof.

In any of the embodiments disclosed herein, the products can be rolling circle amplification (RCA) products and can be detected by: contacting the biological sample with one or more detectably-labeled probes that directly or indirectly hybridize to the RCA products, and dehybridizing the one or more detectably-labeled probes from the RCA products, optionally the contacting and dehybridizing steps can be repeated with the one or more detectably-labeled probes and/or one or more other detectably-labeled probes that directly or indirectly hybridize to the RCA products.

In any of the embodiments disclosed herein, the products can be rolling circle amplification (RCA) products and can be detected by: contacting the biological sample with one or more intermediate probes that directly or indirectly hybridize to the RCA products, the one or more intermediate probes can be detectable using one or more detectably-labeled probes, and dehybridizing the one or more intermediate probes and/or the one or more detectably-labeled probes from the RCA products, optionally the contacting and dehybridizing steps can be repeated with the one or more intermediate probes, the one or more detectably-labeled probes, one or more other intermediate probes, and/or one or more other detectably-labeled probes.

In any of the embodiments disclosed herein, the one or more intermediate probes can each comprise a sequence that hybridizes to one of the RCA products and one or more overhangs that hybridize to a detectably-labeled probe but not to the RCA product.

In any of the embodiments disclosed herein, the method can comprise: (i) contacting the biological sample with detectable probes that hybridize to the target-specific barcode sequence or complement thereof; (ii) imaging the biological sample to detect the plurality of signals of step (b); (iii) optionally removing the detectable probes from the target-specific barcode sequence or complement thereof; (iv) contacting the biological sample with detectable probes that hybridize to the first and second probe-resolution barcode sequences or complements thereof; (v) imaging the biological sample to detect the signal associated with the first probe-resolution barcode sequence in a first detection channel; (vi) imaging the biological sample to detect the signal associated with the second probe-resolution barcode sequence in a second detection channel that is different from the first detection channel; and (vii) optionally removing the detectable probes from the first and second probe-resolution barcode sequences or complements thereof.

In any of the embodiments disclosed herein, the detectable probes that hybridize to the target-specific barcode sequence or complement thereof can comprise intermediate probes that hybridize to the target-specific barcode sequence or complement thereof and detectably-labeled probes that hybridize to the intermediate probes.

In any of the embodiments disclosed herein, the detectable probes that hybridize to the first and second probe-resolution barcode sequences or complements thereof can comprise intermediate probes that hybridize to the first and second probe-resolution barcode sequences or complements thereof and detectably-labeled probes that hybridize to the intermediate probes.

In any of the embodiments disclosed herein, the detectable probes that hybridize to the target-specific barcode sequence or complement thereof can be directly or indirectly labeled with a fluorescent label that is different from fluorescent labels of the detectable probes that hybridize to the first and second probe-resolution barcode sequences or complements thereof. In any of the embodiments disclosed herein, the method may not comprise removing the detectable probes from the target-specific barcode sequence or complement thereof. In any of the embodiments disclosed herein, detecting of detectable probes that hybridize to the target-specific barcode sequence or complement thereof and detectable probes that hybridize to the first and second probe-resolution barcode sequences or complements thereof can be performed simultaneously by contacting the biological sample with: detectable probes that hybridize to the target-specific barcode sequence or complement thereof, and detectable probes that hybridize to the first and second probe-resolution barcode sequences or complements thereof. In any of the embodiments disclosed herein, imaging the biological sample to detect the plurality of signals from the detectable probes that hybridize to the target-specific barcode sequence or complement thereof and imaging the biological sample to detect signals associated with the first and second probe-resolution barcode sequence can be performed in any order. In any of the embodiments disclosed herein, the detectable probes that hybridize to the target-specific barcode sequence or complement thereof and the detectable probes that hybridize to the first and second probe-resolution barcode sequences or complements thereof can be removed from the biological sample after imaging the biological sample to detect the signal associated with the second probe-resolution barcode.

In any of the embodiments disclosed herein, the detectable probes that hybridize to the target-specific barcode sequence or complement thereof can be directly or indirectly labeled with a fluorescent label that is detectable in the same fluorescent channel as a fluorescent label of the detectable probes that hybridize to the first and second probe-resolution barcode sequences or complements thereof. In any of the embodiments disclosed herein, the method can comprise removing the detectable probes from the target-specific barcode sequence or complement thereof. In any of the embodiments disclosed herein, imaging the biological sample to detect signals associated with the first and second probe-resolution barcode sequence can be performed in any order.

In any of the embodiments disclosed herein, between imaging the biological sample to detect signals associated with the first and second probe-resolution barcode sequence, the method may not comprise contacting the biological sample with a probe or removing the probe.

In any of the embodiments disclosed herein, the step of contacting the biological sample with detectable probes that hybridize to the target-specific barcode sequence or complement thereof, the step of imaging the biological sample to detect the plurality of signals associated with the target-specific barcode sequences, and the optional step of removing the detectable probes can be performed prior to the step of contacting the biological sample with detectable probes that hybridize to the first and second probe-resolution barcode sequences or complements thereof, the step of imaging the biological sample to detect the signal associated with the first probe-resolution barcode sequence in the first detection channel, and the step of imaging the biological sample to detect the signal associated with the second probe-resolution barcode sequence in the second detection channel. Alternatively, in any of the embodiments disclosed herein, the step of contacting the biological sample with detectable probes that hybridize to the target-specific barcode sequence or complement thereof, the step of imaging the biological sample to detect the plurality of signals associated with the target-specific barcode sequences, and the optional step of removing the detectable probes can be performed after the step of contacting the biological sample with detectable probes that hybridize to the first and second probe-resolution barcode sequences or complements thereof, the step of imaging the biological sample to detect the signal associated with the first probe-resolution barcode sequence in the first detection channel, and the step of imaging the biological sample to detect the signal associated with the second probe-resolution barcode sequence in the second detection channel.

In any of the embodiments disclosed herein, the method can further comprise repeating any one or more of the contacting step (with detectable probes that hybridize to the target-specific barcode sequence or complement thereof), the imaging step (to detect the plurality of signals), the optional removing step (removing the detectable probes from the target-specific barcode sequence or complement thereof), the contacting step (with detectable probes that hybridize to the first and second probe-resolution barcode sequences or complements thereof), the imaging step (to detect the signal associated with the first probe-resolution barcode sequence in the first detection channel), the imaging step (to detect the signal associated with the second probe-resolution barcode sequence in the second detection channel), and the optional removing step (removing the detectable probes from the first and second probe-resolution barcode sequences or complements thereof) one or more times, each time with a different plurality of detectable probes that hybridize to the target-specific barcode sequence or complement thereof, and/or with the same or a different plurality of detectable probes that hybridize to the first and second probe-resolution barcode sequences or complements thereof.

In some embodiments, the signal associated with the first probe-resolution barcode sequence and the signal the signal associated with the second probe-resolution barcode sequence can be detected at the same location in the biological sample. In some embodiments, the signal associated with the first probe-resolution barcode sequence and the signal the signal associated with the second probe-resolution barcode sequence can be detected at different locations in the biological sample. In some embodiments, the method can further comprise registering images of the imaging steps for detecting the plurality of signals associated with the target-specific barcode sequences, the signal associated with the first probe-resolution barcode sequence, and the signal associated with the second probe-resolution barcode sequence. In some embodiments, the plurality of signals associated with the target-specific barcode sequences, the signal associated with the first probe-resolution barcode sequence, and the signal associated with the second probe-resolution barcode sequence can be associated using the registered images. In some embodiments, the plurality of signals associated with the target-specific barcode sequences can comprise overlapping signals at the same location or at adjacent locations in the biological sample. In some embodiments, each overlapping signal can be associated with the signal associated with the first probe-resolution barcode sequence or the signal associated with the second probe-resolution barcode sequence but not both, thereby resolving the overlapping signals.

In some embodiments, disclosed herein is a method for analyzing a biological sample, comprising: (a) contacting the biological sample with a plurality of circular or circularizable probes comprising a first circular or circularizable probe and a second circular or circularizable probe, wherein the first circular or circularizable probe comprises a target-specific barcode sequence and a first probe-resolution barcode sequence, and the second circular or circularizable probe comprises the target-specific barcode sequence and a second probe-resolution barcode sequence, and wherein the plurality of circular or circularizable probes hybridize to different nucleic acid molecules in the biological sample, and the target-specific barcode sequence corresponds to a target nucleic acid; (b) generating rolling circle amplification (RCA) products of the first and second circular or circularizable probes; (c) contacting the biological sample with detectable probes that hybridize to the RCA products at the complement of the target-specific barcode sequence; (d) detecting signals associated with the target-specific barcode sequence; (e) contacting the biological sample with detectable probes that hybridize to the RCA products at the complement of the first probe-resolution barcode sequence and with detectable probes that hybridize to the RCA products at the complement of the second probe-resolution barcode sequence; and (f) detecting, in separate detection channels, a signal associated with the first probe-resolution barcode sequence and a signal associated with the second probe-resolution barcode sequence.

In any of the embodiments disclosed herein, the target nucleic acid can be DNA or RNA. In any of the embodiments disclosed herein, the target nucleic acid can be genomic DNA, an mRNA, a cDNA, or a reporter oligonucleotide of a probe that targets a target analyte in the biological sample. In any of the embodiments disclosed herein, the first and second circular or circularizable probes can hybridize to different molecules of the same target nucleic acid.

In any of the embodiments disclosed herein, the target-specific barcode sequence can be a first target-specific barcode sequence, the target nucleic acid can be a first target nucleic acid, and the plurality of circular or circularizable probes can further comprise one or more circular or circularizable probes each comprising a second target-specific barcode sequence corresponding to a second target nucleic acid distinct from the first target nucleic acid. In any of the embodiments disclosed herein, the plurality of circular or circularizable probes can comprise a first circular or circularizable probe comprising the second target-specific barcode sequence and the first probe-resolution barcode sequence, and a second circular or circularizable probe comprising the second target-specific barcode sequence and the second probe-resolution barcode sequence.

In any of the embodiments disclosed herein, the detectable probes can comprise fluorescently labeled probes that hybridize to the RCA products. In any of the embodiments disclosed herein, the detectable probes can comprise intermediate probes that hybridize to the RCA products and fluorescently labeled probes that in turn hybridize to the intermediate probes.

In any of the embodiments disclosed herein, the signals associated with the target-specific barcode sequence may comprise overlapping signals that are not spatially resolved into individual puncta, e.g., a signal associated with the target-specific barcode sequence is not spatially resolved from one or more other signals associated with the target-specific barcode sequence. In any of the embodiments disclosed herein, the signal associated with the first probe-resolution barcode sequence can be detected in a first detection channel and spatially resolved from other signals detected in the first detection channel. In any of the embodiments disclosed herein, the signal associated with the second probe-resolution barcode sequence can be detected in a second detection channel and spatially resolved from other signals detected in the second detection channel. In any of the embodiments disclosed herein, one or both of the spatially resolved signal associated with the first probe-resolution barcode sequence and the spatially resolved signal associated with the second probe-resolution barcode sequence can each correspond to a signal that is not spatially resolved in the detection of signals associated with the target-specific barcode sequence.

In some aspects, disclosed herein is a method for analyzing a biological sample, which can comprise: (a) contacting the biological sample with a plurality of probes which each comprising a target specific barcode sequence associated with a target analyte, a first probe of the plurality of probes can comprise a first probe-resolution barcode sequence, associated with a first species of organism and a second probe of the plurality of probes which can comprise a second probe-resolution barcode sequence associated with a second species of organism, and where in the first probe can target a first nucleic acid sequence of the target analyte of the first species of organism and the second probe can target a second nucleic acid sequence of the target analyte of the second species of organism, and the target-specific barcode sequence can correspond to the target analyte; (b) detecting a plurality of signals associated with the target-specific barcode sequences of the plurality of probes; (c1) detecting a signal associated with the first probe-resolution barcode sequence; and (c2) detecting a signal associate with the second probe-resolution barcode sequence, wherein the signals of steps (c1) and (c2) are associated with the target analyte.

In some embodiments, the first nucleic acid sequence and the second nucleic acid sequence can be homologs of the target analyte in the first and second species of organism respectively. In any of the embodiments disclosed herein, the first and second probes can be circular or circularizable probes or probe sets. In any of the embodiments disclosed herein, the target nucleic acid can be DNA or RNA. In any of the embodiments disclosed herein, the target nucleic acid can be a genomic DNA, an mRNA, a cDNA, or a reporter oligonucleotide of a probe that targets a target analyte in the biological sample.

In any of the embodiments disclosed herein, the method can comprise contacting the biological sample with detectable probes that hybridize to the target-specific barcode sequence or complements thereof; and contacting the biological sample with detectable probes that hybridize to the first probe-resolution barcode sequence or complement thereof and with detectable probe that hybridize to the second probe-resolution barcode sequence or complement thereof.

In any of the embodiments disclosed herein, the signal associated with the first probe-resolution barcode sequence and the signal associated with the second probe-resolution barcode sequence can be detected in separate detection channels.

In some aspects, disclosed herein is a kit for analyzing a biological sample, comprising a plurality of probes each comprising a target-specific barcode sequence, wherein a first probe of the plurality of probes comprises a first probe-resolution barcode sequence and a second probe of the plurality of probes comprises a second probe-resolution barcode sequence, and wherein the plurality of probes target different molecules of a target analyte (e.g., a target nucleic acid) in the biological sample, and the target-specific barcode sequence corresponds to target analyte. In some aspects, the kit further comprises detectable probes that directly or indirectly bind to the target-specific barcode sequence or complement thereof. In any of the embodiments disclosed herein, the kit may further comprise detectable probes that directly or indirectly bind to the first probe-resolution barcode sequence or complement thereof. In any of the embodiments disclosed herein, the kit may further comprise detectable probes that directly or indirectly bind to the second probe-resolution barcode sequence or complement thereof.

In some embodiments, a kit for analyzing a biological sample can comprise a plurality of circular or circularizable probes comprising a first circular or circularizable probe and a second circular or circularizable probe, wherein the first circular or circularizable probe comprises a target-specific barcode sequence and a first probe-resolution barcode sequence, and a second circular or circularizable probe comprises the target-specific barcode sequence and a second probe-resolution barcode sequence, and wherein the plurality of circular and circularizable probes hybridize to different nucleic acid molecules in the biological sample, and the target-specific barcode sequence corresponds to a target nucleic acid. In some aspects, the kit may further comprise a first intermediate probe that hybridizes to the complement of the target-specific barcode sequence and a first fluorescently labeled probe that hybridizes to the first intermediate probe. In any of the embodiments disclosed herein, the kit may further comprise a second intermediate probe that hybridizes to the complement of the first probe-resolution barcode sequence and a second fluorescently labeled probe that hybridizes to the second intermediate probe. In any of the embodiments disclosed herein, the kit may further comprise a third intermediate probe that hybridizes to the complement of the second probe-resolution barcode sequence and a third fluorescently labeled probe that hybridizes to the third intermediate probe. In any of the embodiments disclosed herein, the second and third fluorescently labeled probes can be detectable in different fluorescent channels. In any of the embodiments disclosed herein, the first fluorescently labeled probe can be detectable in the same fluorescent channel as the second fluorescently labeled probe or the third fluorescently labeled probe, and the first fluorescently labeled probe may be removed from the biological sample prior to detection of the second and/or third fluorescently labeled probes. Alternatively, in any of the embodiments disclosed herein, the first fluorescently labeled probe can be detectable in a different fluorescent channel from the second fluorescently labeled probe or the third fluorescently labeled probe. In such cases, the first fluorescently labeled probe do not need to but may be removed from the biological sample prior to detection of the second and/or third fluorescently labeled probes.

In any of the embodiments disclosed herein, the target-specific barcode sequence can be a first target-specific barcode sequence, the target nucleic acid can be a first target nucleic acid, and the plurality of circular or circularizable probes can further comprise one or more circular or circularizable probes each comprising a second target-specific barcode sequence corresponding to a second target nucleic acid distinct from the first target nucleic acid. In some embodiments, the kit can further comprise the plurality of circular or circularizable probes which can comprise a first circular or circularizable probe comprising the second target-specific barcode sequence and the first probe-resolution barcode sequence, and a second circular or circularizable probe comprising the second target-specific barcode sequence and the second probe-resolution barcode sequence.

### BRIEF DESCRIPTION OF THE DRAWINGS

The patent or application file contains at least one drawing executed in color. Copies of this patent or patent application publication with color drawings will be provided by the Office upon request and payment of the necessary fee.

The drawings illustrate certain embodiments of the features and advantages of this disclosure. These embodiments are not intended to limit the scope of the appended claims in any manner.

FIGS. 1A-1C show schematics illustrating the probe-resolution barcode (high-resolution tag) strategy. FIG. 1A shows four padlock probes for detecting Gene X. All four padlock probes may contain a common target-specific barcode sequence (e.g., gene-specific barcode sequence) corresponding to Gene X and each of the probes can contain a probe-resolution barcode sequence (also referred to as HR Tag 1, HR Tag 2, HR Tag 3, and HR Tag 4) that can be used to distinguish one Gene X probe from the other three Gene X probes. FIG. 1B shows the probe-resolution barcode sequences can be detected by their respective detectable probes, such as an intermediate probe (e.g., the L-shaped probe shown in the figure) and a fluorescently labelled probe recognizing the intermediate probe. FIG. 1C shows a probe-resolution barcode sequence can be common among probes targeting different analytes, e.g., a padlock probe for Gene X and a padlock probe for Gene Y can share the same probe-resolution barcode sequence while comprising different gene specific barcode sequences for Gene X and Gene Y, respectively.

FIG. 2A shows an illustration of the probe-resolution barcode strategy. Signals are initially detected with detectable probes for a target-specific barcode sequence in RCA products corresponding to a gene of interest, where some signals are overlapping and cause optical crowding (FIG. 2A, left). The RCA products in the sample are detected with detectable probes for the probe-resolution barcode sequences, such that signals associated with different subsets of the RCA products corresponding to the same gene of interest can be detected in different color channels (Channels 1-4) (FIG. 2A, middle). Signal spots from different channels are superimposed to illustrate that higher resolution can be achieved by detecting probe-resolution barcode sequences (FIG. 2A, right). The color channel used to detect the target-specific barcode can be same as or different from any one of Channels 1-4. The probe-resolution barcode sequences can be detected in any order, as indicated by bi-directional arrows between the images in different channels.

FIGS. 2B-2C show the in situ detection of a highly expressed gene Malat-1 on fresh frozen mouse brain tissue section. FIG. 2B shows fluorescence images of a representative cell in the tissue section showing gene-specific barcode detection in one fluorescent channel and the probe-resolution barcode detection in four separate fluorescent channels. FIG. 2C shows the total number of resolved RCA products obtained for Malat-1, quantified after target-specific barcode detection and subsequently with the probe-resolution barcode detection.

FIGS. 3A-3B show the in situ detection of human and mouse Malat-1 on samples from a PDX mouse model of Diffuse Intrinsic Pontine Glioma (DIPG).

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

When preparing in situ sequencing libraries of highly expressed or abundant targets (e.g., genes), the dynamic range can be hindered by optical crowding of signals. Optical crowding can be the result of many locally amplified probes in close proximity, impeding the precise quantification of the expression levels of highly expressed genes. For instance, for highly expressed genes, the amplified probes are highly likely to be overlapping or in very close proximity with each other. These amplified probes will in turn produce overlapping or very close signals that are detected as one single signal using common optical detection method. As a result, the total number of detected signals would be reduced, causing the detected expressing level to be lower than that is present in the sample. Thus, there is a need for methods and compositions for precise quantification of the expression levels of highly expressed genes.

In some aspects, the present disclosure provides methods and compositions for precise quantification of the expression levels of highly expressed genes. In some embodiments, in situ sequencing libraries and methods of barcode detection, e.g., by sequential probe hybridization or sequencing-by-hybridization (SBH) reactions, are provided. In some embodiments, the compositions and methods disclosed herein allow resolution of highly multiplexed reactions, in which one or several highly expressed genes cause optical crowding and limit the dynamic range. In some aspects, provided herein are methods and compositions for detecting the expression levels of highly expressed genes using multiple probes (e.g., padlock probes) to target a single gene, wherein each probe contains an individual probe-resolution barcode sequence (“high-resolution tag”). By labelling and detecting these individual probe-resolution barcode sequences using their corresponding detectable probes (e.g., SBH read-out probes), each gene can be detected in different and multiple fluorescent channels. In some embodiments, different subsets of amplification (e.g., RCA) products associated with the same gene can be detected in different fluorescent channels, thus overcoming optical crowding and increasing the dynamic range of either a highly multiplexed amplification (e.g., RCA) reaction or a sample with one or several highly expressed genes. Thus, in some aspects, the compositions and methods herein are particularly useful for analyzing a sample with high amplification (e.g., RCA) product density, for instance, by detecting subsets of RCA products in separate fluorescent channels to better resolve signals associated with the RCA products.

In some embodiments, multiple padlock probes containing different probe-resolution barcode sequences can be used to target a gene known or suspected to be highly expressed is a sample. In some embodiments, multiple genes known or suspected to be highly expressed in a sample can each be targeted by multiple padlock probes containing different probe-resolution barcode sequences. In some embodiments, all of the genes to be detected in a sample are targeted by multiple padlock probes containing different probe-resolution barcode sequences. In some embodiments, the same set of different probe-resolution barcode sequences are used in the padlock probes for different genes and gene specific barcodes are used to differentiate padlock probes for one gene from padlock probes for a different gene. In some embodiments, the multiple padlock probes for a gene can bind to different regions of the gene. In some embodiments, the multiple padlock probes containing different probe-resolution barcode sequences are used as template to generate RCA products in situ. In some embodiments, detectable probes for each different probe-resolution barcode sequence can be used to hybridize to the probe-resolution barcode sequences or complements thereof in the RCA products.

In some aspects, provided herein are methods and compositions for detecting the species origin using multiple padlock probes to target a single gene, wherein each probe contains an individual probe-resolution barcode sequence (“species-specific tag”). By labelling and detecting these individual probe-resolution barcode sequences using their corresponding detectable probes (e.g., species-specific readout probes), each gene can be detected in different and multiple fluorescent channels. In some embodiments, different subsets of amplification products associated with the same gene can be detected in different fluorescent channels, for example by detecting a probe-resolution barcode sequence (“species-specific tag A”) of a first probe associated with a first species in a first fluorescent channel and detecting a probe-resolution barcode sequence (“species-specific tag B”) of a second probe associated with a second species in another fluorescent channel.

In some embodiments, provided herein is a method for analyzing a biological sample, comprising contacting the biological sample with: (i) a first probe comprising a first target-specific barcode sequence and a first probe-resolution barcode sequence, and (ii) a second probe comprising a second target-specific barcode sequence and a second probe-resolution barcode sequence. In some embodiments, the first and second target-specific barcode sequences are identical. In some embodiments, the first and second probes target a target nucleic acid (e.g., genomic DNA, mtDNA, mRNA, cDNA, RCA product, or oligonucleotide conjugated to a binder such as an antibody) in the biological sample. In some embodiments, the first and second target-specific barcode sequences correspond to the nucleic acid molecule, and the first and second probe-resolution barcode sequences distinguish the first and second probes from each other. In some embodiments, the first and/or second probes are circular probes. In some embodiments, the first and/or second probes are circularizable probes, such as padlock probes. In some embodiments, the first and second target-specific barcode sequences are identical barcode sequences. In some embodiments, the first and second target-specific barcode sequences are different in sequence and yet both correspond to the same nucleic acid present or suspected of being present in the biological sample. In any of the embodiments herein, the first probe-resolution barcode sequence can be common among a first plurality of probes each targeting a different analyte, such as distinct nucleic acid sequences of interest. In any of the embodiments herein, the second probe-resolution barcode sequence can be common among a second plurality of probes each targeting a different analyte, such as distinct nucleic acid sequences of interest. In some embodiments, the first plurality of probes and the second plurality of probes can target the same or different analytes.

In any of the embodiments herein, the method can further comprise contacting the biological sample with detectable probes that hybridize to the first and second target-specific barcode sequences or complements thereof. In any of the embodiments herein, the method can further comprise detecting signals associated with the target-specific barcode sequence in the biological sample to provide signals indicative of the nucleic acid molecule.

In any of the embodiments herein, the method can further comprise contacting the biological sample with detectable probes that hybridize to the first and second probe-resolution barcode sequences or complements thereof. In any of the embodiments herein, the method can further comprise detecting signals associated with the first and second probe-resolution barcode sequences in the biological sample to provide signals indicative of the first and second probes.

In some embodiments, signals associated with the target-specific barcode sequence at multiple locations in the biological sample are detected simultaneously, e.g., in the same microscope field of view and in the same fluorescent channel, while signals associated with the first and second probe-resolution barcode sequences at the multiple locations are not all detected simultaneously. For instance, signals associated with the first probe-resolution barcode sequences are detected in a fluorescent channel, while signals associated with the second probe-resolution barcode sequences are detected in a different fluorescent channel. The microscope field of view preferably remain the same between the different fluorescent channels, but the field of view may change provided that the same location in the sample can be tracked. For instance, signals associated with the same amplification (e.g., RCA) product but detected in the different fluorescent channel can be correlated with each other. In some aspects, while signals indicative of the target nucleic acid (e.g., a highly expressed gene transcript) may be detected as overlapping spots by detecting a target-specific barcode sequence or complement thereof in multiple amplification (e.g., RCA) products, signals associated with each particular probe-resolution barcode sequence or complement thereof correspond to only a subset of the multiple amplification (e.g., RCA) products. As such, overlapping signals indicative of the target nucleic acid can be resolved by separately detecting the signals spread across different detection channels. In some cases, detecting of the signals across different channels makes it easier to resolve signal spots in each detection channel as well as signal spots in different detection channels that would otherwise be overlapping. In some cases, detecting of the signals across different channels allows identification of subsets of the signals associated with the same target analyte to be associated with a particular origin (e.g., species origin such as mouse or human).

### II. Samples, Analytes, and Target Sequences

A. Samples

A sample disclosed herein can be or be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally comprises cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can comprise any number of macromolecules, for example, cellular macromolecules and organelles (e.g., mitochondria and nuclei). The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining

To facilitate visualization, biological samples can be stained using a wide variety of stains and staining techniques. In some embodiments, for example, a sample can be stained using any number of stains, including but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, propidium iodide, rhodamine, or safranine.

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay round. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of species (such as probes) into the sample. If a sample is not permeabilized sufficiently, the amount of species (such as probes) in the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some embodiments, one or more nucleic acid probes can be used to hybridize to a target nucleic acid (e.g., cDNA or RNA molecule, such as an mRNA) and ligated in a templated ligation reaction (e.g., RNA-templated ligation (RTL) or DNA-templated ligation (e.g., on cDNA)) to generate a product for analysis. In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte (e.g., target analyte) comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being detectable.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

(a) Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences and can be modified with a probe resolution barcode sequence as described in Section IV. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ hybridization) probe set, and RNA-templated ligation probes. The specific probe or probe set design can vary. In some embodiments, the probe or probe set comprises a circularizable probe or probe set.

(b) Ligation

In some embodiments, a product of an endogenous analyte and/or a labelling agent is a ligation product. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 2019/0055594, which is hereby incorporated by reference in its entirety. In some embodiments, provided herein is a multiplexed proximity ligation assay. See, e.g., U.S. Pat. Pub. 2014/0194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 2016/0108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. This selectively reduces the concentration of annealed mismatched substrates (expected to have a slightly lower Tm around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(c) Primer Extension and Amplification

In some embodiments, a product is a primer extension product of an analyte, a labelling agent, a probe or probe set bound to the analyte (e.g., a padlock probe bound to genomic DNA, mRNA, or cDNA), or a probe or probe set bound to the labelling agent (e.g., a padlock probe bound to one or more reporter oligonucleotides from the same or different labelling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) may include linear RCA, a branched RCA, a dendritic RCA, or any combination thereof. See, e.g., Baner et al, Nucleic Acids Research, 26:5073-5078, 1998; Lizardi et al, Nature Genetics 19:226, 1998; Mohsen et al., Acc Chem Res. 2016 November 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:e118, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801. Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, US 2016/0024555, US 2018/0251833, US 2017/0219465, U.S. Pat. Nos. 10,138,509, 10,494,662, 11,078,520, 11,299,767, 10,266,888, 11,118,220, US 2021/0363579, and US 2021/0215581, all of which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. RCA-based detection systems can be used for the detection of different analytes, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

C. Target Sequences

A target sequence for the probes disclosed herein may be comprised in any analyte disclose herein, including an endogenous analyte (e.g., a viral or cellular nucleic acid), a labelling agent, or a product of an endogenous analyte and/or a labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, barcodes (e.g., primary and/or secondary barcode sequences, target-specific and/or probe-resolution barcode sequences as described in Section IV) can be analyzed (e.g., detected or sequenced) using any suitable methods or techniques, including those described herein. In some embodiments, the methods provided herein can include analyzing the barcodes by sequential hybridization and detection with a plurality of labelled probes (e.g., detection oligonucleotides).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4′ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., US 2019/0055594 and US 2021/0164039 which are hereby incorporated by reference in their entirety.

### III. Nucleic Acid Probes

Disclosed herein in some aspects are nucleic acid probes and/or probe sets that are introduced into a cell or used to otherwise contact a biological sample such as a tissue sample. The probes may comprise any of a variety of entities that can hybridize to a nucleic acid, typically by Watson-Crick base pairing, such as DNA, RNA, LNA, PNA, etc. The nucleic acid probe typically contains a targeting sequence that is able to directly or indirectly bind to at least a portion of a target nucleic acid. The nucleic acid probe may be able to bind to a specific target nucleic acid (e.g., an mRNA, or other nucleic acids as discussed herein). In some embodiments, the nucleic acid probes may be detected using a detectable label, and/or by using secondary nucleic acid probes able to bind to the nucleic acid probes. In some embodiments, the nucleic acid probes (e.g., primary probes and/or secondary probes) are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease or exonuclease for cleavage or digestion).

In some embodiments, more than one type of primary nucleic acid probes may be contacted with a sample, e.g., simultaneously or sequentially in any suitable order, such as in sequential probe hybridization/unhybridization cycles. In some embodiments, the primary probes may comprise circular probes and/or circularizable probes (such as padlock probes). In some embodiments, more than one type of secondary nucleic acid probes may be contacted with a sample, e.g., simultaneously or sequentially in any suitable order, such as in sequential probe hybridization/unhybridization cycles. In some embodiments, the secondary probes may comprise probes that bind to a product (e.g., an RCA product) of a primary probe targeting an analyte. In some embodiments, more than one type of higher order nucleic acid probes may be contacted with a sample, e.g., simultaneously or sequentially in any suitable order, such as in sequential probe hybridization/unhybridization cycles. In some embodiments, more than one type of detectably labeled nucleic acid probes may be contacted with a sample, e.g., simultaneously or sequentially in any suitable order, such as in sequential probe hybridization/unhybridization cycles. In some embodiments, the detectably labeled probes may comprise probes that bind to one or more primary probes, one or more secondary probes, one or more higher order probes, one or more intermediate probes between a primary/second/higher order probes, and/or one or more detectably or non-detectably labeled probes (e.g., as in the case of a hybridization chain reaction (HCR), a branched DNA reaction (bDNA), or the like). In some embodiments, at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid probes (e.g., primary, secondary, higher order probes, and/or detectably labeled probes) can be contacted with a sample, e.g., simultaneously or sequentially in any suitable order. Between any of the probe contacting steps disclosed herein, the method may comprise one or more intervening reactions and/or processing steps, such as modifications of a target nucleic acid, modifications of a probe or product thereof (e.g., via hybridization, ligation, extension, amplification, cleavage, digestion, branch migration, primer exchange reaction, click chemistry reaction, crosslinking, attachment of a detectable label, activating photo-reactive moieties, etc.), removal of a probe or product thereof (e.g., cleaving off a portion of a probe and/or unhybridizing the entire probe), signal modifications (e.g., quenching, masking, photo-bleaching, signal enhancement (e.g., via FRET), signal amplification, etc.), signal removal (e.g., cleaving off or permanently inactivating a detectable label), crosslinking, de-crosslinking, and/or signal detection.

The target-binding sequence (sometimes also referred to as the targeting region/sequence or the recognition region/sequence) of a probe may be positioned anywhere within the probe. For instance, the target-binding sequence of a primary probe that binds to a target nucleic acid can be 5′ or 3′ to any barcode sequence in the primary probe. Likewise, the target-binding sequence of a secondary probe (which binds to a primary probe or complement or product thereof) can be 5′ or 3′ to any barcode sequence in the secondary probe. In some embodiments, the target-binding sequence may comprise a sequence that is substantially complementary to a portion of a target nucleic acid. In some embodiments, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

The target-binding sequence of a primary nucleic acid probe may be determined with reference to a target nucleic acid (e.g., a cellular RNA or a reporter oligonucleotide of a labelling agent for a cellular analyte) that is present or suspected of being present in a sample. In some embodiments, more than one target-binding sequence can be used to identify a particular analyte comprising or associated with a target nucleic acid. The more than one target-binding sequence can be in the same probe or in different probes. For instance, multiple probes can be used, sequentially and/or simultaneously, that can bind to (e.g., hybridize to) different regions of the same target nucleic acid. In other examples, a probe may comprise target-binding sequences that can bind to different target nucleic acid sequences, e.g., various intron and/or exon sequences of the same gene (for detecting splice variants, for example), or sequences of different genes, e.g., for detecting a product that comprises the different target nucleic acid sequences, such as a genome rearrangement (e.g., inversion, transposition, translocation, insertion, deletion, duplication, and/or amplification).

After contacting the nucleic acid probes with a sample, the probes may be directly detected by determining detectable labels (if present), and/or detected by using one or more other probes that bind directly or indirectly to the probes or products thereof. The one or more other probes may comprise a detectable label. For instance, a primary nucleic acid probe can bind to a target nucleic acid in the sample, and a secondary nucleic acid probe can be introduced to bind to an amplification product of the primary nucleic acid probe, where the secondary nucleic acid probe or a product thereof can then be detected using detectably labeled probes. Higher order probes that directly or indirectly bind to the secondary nucleic acid probe or product thereof may also be used, and the higher order probes or products thereof can then be detected using detectably labeled probes.

In some embodiments, the detection may be spatial, e.g., in two or three dimensions. In some embodiments, the detection may be quantitative, e.g., the amount or concentration of a primary nucleic acid probe (and of a target nucleic acid) may be determined. In some embodiments, the primary probes, secondary probes, higher order probes, and/or detectably labeled probes may comprise any of a variety of entities able to hybridize a nucleic acid, e.g., DNA, RNA, LNA, and/or PNA, etc., depending on the application.

A secondary nucleic acid probe may contain a recognition sequence able to bind to or hybridize with a primary nucleic acid probe or a product thereof, e.g., at a barcode sequence or portion(s) thereof of the primary nucleic acid probe, or at a complement of the barcode sequence or portion(s) thereof (e.g., in the case of the secondary probe hybridizing to an RCA product of the primary probe). In some embodiments, a secondary nucleic acid probe may bind to a combination of barcode sequences (which may be continuous or spaced from one another) in a primary nucleic acid probe or a product thereof. In some embodiments, the binding is specific, or the binding may be such that a recognition sequence preferentially binds to or hybridizes with only one of the barcode sequences or complements thereof that are present. The secondary nucleic acid probe may also contain one or more detectable labels. If more than one secondary nucleic acid probe is used, the detectable labels may be the same or different.

The recognition sequences may be of any length, and multiple recognition sequences in the same or different secondary nucleic acid probes may be of the same or different lengths. If more than one recognition sequence is used, the recognition sequences may independently have the same or different lengths. For instance, the recognition sequence may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence may be no more than 48, no more than 40, no more than 32, no more than 24, no more than 16, no more than 12, no more than 10, no more than 8, or no more than 6 nucleotides in length. Combinations of any of these are also possible, e.g., the recognition sequence may have a length of between 5 and 8, between 6 and 12, or between 7 and 15 nucleotides, etc. In one embodiment, the recognition sequence is of the same length as a barcode sequence or complement thereof of a primary nucleic acid probe or a product thereof. In some embodiments, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to the barcode sequence or complement thereof.

In some embodiments, a nucleic acid probe, such as a primary or a secondary nucleic acid probe, may also comprise 1, 2, 3, 4, 5, 6, 7, 8, 9, 10, 11, 12, 13, 14, 15 or more, 20 or more, 32 or more, 40 or more, or 50 or more barcode sequences. The barcode sequences may be any target-specific barcode sequence or any probe-resolution barcode sequence as described herein. The barcode sequences may be positioned anywhere within the nucleic acid probe. If more than one barcode sequences are present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of nucleic acid probes is less than the number of distinct targets (e.g., nucleic acid analytes and/or protein analytes) of the nucleic acid probes, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes.

As an illustrative example, a first probe may contain a first target-binding sequence, a first barcode sequence, and a second barcode sequence, while a second, different probe may contain a second target-binding sequence (that is different from the first target-binding sequence in the first probe), the same first barcode sequence as in the first probe, but a third barcode sequence instead of the second barcode sequence. Such probes may thereby be distinguished by determining the various barcode sequence combinations present or associated with a given probe at a given location in a sample.

In some embodiments, the nucleic acid probes disclosed herein may be made using only 2 or only 3 of the 4 bases, such as leaving out all the “G”s and/or leaving out all of the “C”s within the probe. Sequences lacking either “G”s or “C”s may form very little secondary structure, and can contribute to more uniform, faster hybridization in certain embodiments.

In some embodiments, a nucleic acid probe disclosed herein may contain a detectable label such as a fluorophore. In some embodiments, one or more probes of a plurality of nucleic acid probes used in an assay may lack a detectable label, while one or more other probes in the plurality each comprises a detectable label selected from a limited pool of distinct detectable labels (e.g., red, green, yellow, and blue fluorophores), and the absence of detectable label may be used as a separate “color.” As such, detectable labels are not required in all cases. In some embodiments, a primary nucleic acid probe (e.g., a padlock probe) disclosed herein lacks a detectable label. While a detectable label may be incorporated into an amplification product of the primary nucleic acid probe, such as via incorporation of a modified nucleotide into an RCA product of a padlock probe, the amplification product in some embodiments is not detectably labeled. In some embodiments, a probe that binds to the primary nucleic acid probe or a product thereof (e.g., a secondary nucleic acid probe that binds to a barcode sequence or complement thereof in the primary nucleic acid probe or product thereof) comprises a detectable label and may be used to detect the primary nucleic acid probe or product thereof. In some embodiments, a secondary nucleic acid probe disclosed herein lacks a detectable label, and a detectably labeled probe that binds to the secondary nucleic acid probe or a product thereof (e.g., at a barcode sequence or complement thereof in the secondary nucleic acid probe or product thereof) can be used to detect the second nucleic acid probe or product thereof. In some embodiments, signals associated with the detectably labeled probes can be used to detect one or more barcode sequences in the secondary probe and/or one or more barcode sequences in the primary probe, e.g., by using sequential hybridization of detectably labeled probes, sequencing-by-ligation, and/or sequencing-by-hybridization. In some embodiments, the barcode sequences (e.g., in the secondary probe and/or in the primary probe) are used to combinatorially encode a plurality of analytes of interest. As such, signals associated with the detectably labeled probes at particular locations in a biological sample can be used to generate distinct signal signatures that each corresponds to an analyte in the sample, thereby identifying the analytes at the particular locations, e.g., for in situ spatial analysis of the sample.

In some embodiments, a nucleic acid probe herein comprises one or more other components, such as one or more primer binding sequences (e.g., to allow for enzymatic amplification of probes), enzyme recognition sequences (e.g., for endonuclease cleavage), or the like. The components of the nucleic acid probe may be arranged in any suitable order.

In some aspects, analytes are targeted by primary probes, which are barcoded through the incorporation of one or more barcode sequences (e.g., sequences that can be detected or otherwise “read”) that are separate from a sequence in a primary probe that directly or indirectly binds the targeted analyte. In some aspects, the primary probes are in turn targeted by secondary probes, which are also barcoded through the incorporation of one or more barcode sequences that are separate from a recognition sequence in a secondary probe that directly or indirectly binds a primary probe or a product thereof. In some embodiments, a secondary probe may bind to a barcode sequence in the primary probe. In some embodiments, a secondary probe may bind to a complement of the barcode sequence in an RCA product of the primary probe. In some embodiments, one set of secondary probes bind to target-specific barcode sequences in the RCA product and a second set of secondary probes bind to probe-resolution barcode sequences in the RCA product. In some aspects, tertiary probes and optionally even higher order probes may be used to target the secondary probes, e.g., at a barcode sequence or complement thereof in a secondary probe or product thereof. In some embodiments, the tertiary probes and/or even higher order probes may comprise one or more barcode sequences and/or one or more detectable labels. In some embodiments, a tertiary probe is a detectably labeled probe that hybridizes to a barcode sequence (or complement thereof) of a secondary probe (or product thereof). In some embodiments, through the detection of signals associated with detectably labeled probes in a sample, the location of one or more analytes in the sample and the identity of the analyte(s) can be determined. In some embodiments, the presence/absence, absolute or relative abundance, an amount, a level, a concentration, an activity, and/or a relation with another analyte of a particular analyte can be analyzed in situ in the sample.

In some embodiments, provided herein are probes, probe sets, and assay methods to couple target nucleic acid detection, signal amplification (e.g., through nucleic acid amplification such as RCA, and/or hybridization of a plurality of detectably labeled probes, such as in hybridization chain reactions and the like), and decoding of the barcodes.

In some aspects, a primary probe (e.g., comprising a target-specific barcode sequence and a probe-resolution barcode sequence as described in Section IV), a secondary probe, and/or a higher order probe can be selected from the group consisting of a circular probe, a circularizable probe, and a linear probe. In some embodiments, a circular probe can be one that is pre-circularized prior to hybridization to a target nucleic acid and/or one or more other probes. In some embodiments, a circularizable probe can be one that can be circularized upon hybridization to a target nucleic acid and/or one or more other probes such as a splint. In some embodiments, a linear probe can be one that comprises a target recognition sequence and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) is non-hybridizing to the target nucleic acid but may hybridize to one another and/or one or more other probes, such as detectably labeled probes.

Specific probe designs can vary depending on the application. For instance, a primary probe, a secondary probe, and/or a higher order probe disclosed herein can comprise a circularizable probe (e.g., padlock probe) that does require gap filling to circularize upon hybridization to a template (e.g., a target nucleic acid and/or a probe such as a splint), a gapped padlock probe (e.g., one that require gap filling to circularize upon hybridization to a template), an L-shaped probe (e.g., one that comprises a target recognition sequence and a 5′ or 3′ overhang upon hybridization to a target nucleic acid or a probe), a U-shaped probe (e.g., one that comprises a target recognition sequence, a 5′ overhang, and a 3′ overhang upon hybridization to a target nucleic acid or a probe), a V-shaped probe (e.g., one that comprises at least two target recognition sequences and a linker or spacer between the target recognition sequences upon hybridization to a target nucleic acid or a probe), a probe or probe set for proximity ligation (such as those described in U.S. Pat. Nos. 7,914,987 and 8,580,504 incorporated herein by reference in their entireties, and probes for Proximity Ligation Assay (PLA) for the simultaneous detection and quantification of nucleic acid molecules and protein-protein interactions), or any suitable combination thereof. In some embodiments, a primary probe, a secondary probe, and/or a higher order probe disclosed herein can comprise a padlock-like probe or probe set. In some embodiments, a nucleic acid probe disclosed herein is part of a SNAIL (Splint Nucleotide Assisted Intramolecular Ligation) probe set, such as one described in US 2019/0055594 or US 2021/0164039 which are incorporated herein by reference in their entireties. In some embodiments, a nucleic acid probe disclosed herein is part of a PLAYR (Proximity Ligation Assay for RNA) probe set, such as one described in US 2016/0108458 which is incorporated herein by reference in its entirety. In some embodiments, a nucleic acid probe disclosed herein is part of a PLISH (Proximity Ligation in situ Hybridization) probe set, such as one described in US 2020/0224243 which is incorporated herein by reference in its entirety. Any suitable combination of the probe designs described herein can be used.

Any suitable circularizable probe or probe set, or indeed more generally circularizable reporter molecules, may be used to generate the RCA template which is used to generate the RCA product. By “circularizable” is meant that the probe or reporter (the RCA template) is in the form of a linear molecule having ligatable ends which may circularized by ligating the ends together directly or indirectly, e.g., to each other, or to the respective ends of an intervening (“gap”) oligonucleotide or to an extended 3′ end of the circularizable RCA template. A circularizable template may also be provided in two or more parts, namely two or more molecules (e.g., oligonucleotides) which may be ligated together to form a circle. When said RCA template is circularizable it is circularized by ligation prior to RCA. Ligation may be templated using a ligation template, and in the case of padlock and molecular inversion probes and such like the target analyte may provide the ligation template, or it may be separately provided. The circularizable RCA template (or template part or portion) will comprise at its respective 3′ and 5′ ends regions of complementarity to corresponding cognate complementary regions (or binding sites) in the ligation template, which may be adjacent where the ends are directly ligated to each other, or non-adjacent, with an intervening “gap” sequence, where indirect ligation is to take place.

In the case of padlock probes, in one embodiment the ends of the padlock probe may be brought into proximity to each other by hybridization to adjacent sequences on a target nucleic acid molecule (such as a target analyte), which acts as a ligation template, thus allowing the ends to be ligated together to form a circular nucleic acid molecule, allowing the circularized padlock probe to act as a template for an RCA reaction. In such an example the terminal sequences of the padlock probe which hybridize to the target nucleic acid molecule will be specific to the target analyte in question, and will be replicated repeatedly in the RCA product. They may therefore act as a marker sequence indicative of that target analyte. Accordingly, it can be seen that the marker sequence in the RCA product may be equivalent to a sequence present in the target analyte itself. Alternatively, a marker sequence (e.g. tag or barcode sequence) may be provided in the non-target complementary parts of the padlock probe. In still a further embodiment, the marker sequence may be present in the gap oligonucleotide which is hybridized between the respective hybridized ends of the padlock probe, where they are hybridized to non-adjacent sequences in the target molecule. Such gap-filling padlock probes are akin to molecular inversion probes.

In some embodiments, similar circular RCA template molecules can be generated using molecular inversion probes. Like padlock probes, these are also typically linear nucleic acid molecules capable of hybridizing to a target nucleic acid molecule (such as a target analyte) and being circularized. The two ends of the molecular inversion probe may hybridize to the target nucleic acid molecule at sites which are proximate but not directly adjacent to each other, resulting in a gap between the two ends. The size of this gap may range from only a single nucleotide in some embodiments, to larger gaps of 100 to 500 nucleotides, or longer, in other embodiments. Accordingly, it is necessary to supply a polymerase and a source of nucleotides, or an additional gap-filling oligonucleotide, in order to fill the gap between the two ends of the molecular inversion probe, such that it can be circularized.

As with the padlock probe, the terminal sequences of the molecular inversion probe which hybridize to the target nucleic acid molecule, and the sequence between them, will be specific to the target analyte in question, and will be replicated repeatedly in the RCA product. They may therefore act as a marker sequence indicative of that target analyte. Alternatively, a marker sequence (e.g. tag or barcode sequence) may be provided in the non-target complementary parts of the molecular inversion probe.

In some embodiments, the probes disclosed herein may be invader probes, e.g., for generating a circular nucleic acid such as a circularized probe. Such probes are of particular utility in the detection of single nucleotide polymorphisms. The detection method of the present disclosure may, therefore, be used in the detection of a single nucleotide polymorphism, or indeed any variant base, in the target nucleic acid sequence. Probes for use in such a method may be designed such that the 3′ ligatable end of the probe is complementary to and capable of hybridizing to the nucleotide in the target molecule which is of interest (the variant nucleotide), and the nucleotide at the 3′ end of the 5′ additional sequence at the 5′ end of the probe or at the 5′ end of another, different, probe part is complementary to the same said nucleotide, but is prevented from hybridizing thereto by a 3′ ligatable end (e.g., it is a displaced nucleotide). Cleavage of the probe to remove the additional sequence provides a 5′ ligatable end, which may be ligated to the 3′ ligatable end of the probe or probe part if the 3′ ligatable end is hybridized correctly to (e.g., is complementary to) the target nucleic acid molecule. Probes designed according to this principle provide a high degree of discrimination between different variants at the position of interest, as only probes in which the 3′ ligatable end is complementary to the nucleotide at the position of interest may participate in a ligation reaction. In one embodiment, the probe is provided in a single part, and the 3′ and 5′ ligatable ends are provided by the same probe. In some embodiments, an invader probe is a padlock probe (an invader padlock or “iLock”), e.g., as described in Krzywkowski et al., Nucleic Acids Research 45, e161, 2017 and US 2020/0224244, which are incorporated herein by reference.

Other types of probe which result in circular molecules which can be detected by RCA and which comprise either a target analyte sequence or a complement thereof have been developed by Olink Bioscience (now Navinci Diagnostics AB) and include the Selector-type probes described in U.S. Pat. No. 10,612,093, which comprise sequences capable of directing the cleavage of a target nucleic acid so as to release a fragment comprising a target sequence from the target analyte and sequences capable of templating the circularization and ligation of the fragment. WO 2016/016452 describes probes which comprise a 3′ sequence capable of hybridizing to a target nucleic acid and acting as a primer for the production of a complement of a target sequence within the target nucleic acid molecule (e.g., by target templated extension of the primer), and an internal sequence capable of templating the circularization and ligation of the extended probe comprising the reverse complement of the target sequence within the target analyte and a portion of the probe. In the case of both such probes, target sequences or complements thereof are incorporated into a circularized molecule which acts as the template for the RCA reaction to generate the RCA product, which consequently comprises concatenated repeats of said target sequence. Again, said target sequence may act as, or may comprise a marker sequence within the RCA product indicative of the target analyte in question. Alternatively, a marker sequence (e.g. tag or barcode sequence) may be provided in the non-target complementary parts of the probes.

In some embodiments, a nucleic acid probe disclosed herein can be pre-assembled from multiple components, e.g., prior to contacting the nucleic acid probe with a target nucleic acid or a sample. In some embodiments, a nucleic acid probe disclosed herein can be assembled during and/or after contacting a target nucleic acid or a sample with multiple components. In some embodiments, a nucleic acid probe disclosed herein is assembled in situ in a sample. In some embodiments, the multiple components can be contacted with a target nucleic acid or a sample in any suitable order and any suitable combination. For instance, a first component and a second component can be contacted with a target nucleic acid, to allow binding between the components and/or binding between the first and/or second components with the target nucleic acid. Optionally a reaction involving either or both components and/or the target nucleic acid, between the components, and/or between either one or both components and the target nucleic acid can be performed, such as hybridization, ligation, primer extension and/or amplification, chemical or enzymatic cleavage, click chemistry, or any combination thereof. In some embodiments, a third component can be added prior to, during, or after the reaction. In some embodiments, a third component can be added prior to, during, or after contacting the sample with the first and/or second components. In some embodiments, the first, second, and third components can be contacted with the sample in any suitable combination, sequentially or simultaneously. In some embodiments, the nucleic acid probe can be assembled in situ in a stepwise manner, each step with the addition of one or more components, or in a dynamic process where all components are assembled together. One or more removing steps, e.g., by washing the sample such as under stringent conditions, may be performed at any point during the assembling process to remove or destabilize undesired intermediates and/or components at that point and increase the chance of accurate probe assembly and specific target binding of the assembled probe.

### IV. In Situ Analysis Using Target-Specific and Probe-Resolution Barcode Sequences

In some embodiments, disclosed herein is a method for analyzing a biological sample, comprising contacting the biological sample with a plurality of probes each comprising a target-specific barcode sequence, wherein a first probe of the plurality of probes comprises a first probe-resolution barcode sequence and a second probe of the plurality of probes comprises a second probe-resolution barcode sequence. In some embodiments, the plurality of probes target a target nucleic acid in the biological sample, and the target-specific barcode sequence corresponds to the target nucleic acid. In some embodiments, the first and second probe-resolution barcode sequences are distinct. In some embodiments, the first and second probe-resolution barcode sequences do not correspond to any particular nucleic acid molecule in the biological sample, but rather distinguish the first probe from the second probe, where both probes correspond to the same nucleic acid molecule.

In some embodiments, the target-specific barcode sequence can be about 5, about 10, about 15, about 20, about 25, about 30, or about 35 nucleotides in length. In some embodiments, the target-specific barcode sequence can be about 10, 11, 12, 13, 14, 15, 16, 17, 18, 19, 20, 21, 22, 23, 24, 25, or more nucleotides in length.

In some embodiments, the first and second probe-resolution barcode sequences can be independently about 3, about 5, about 10, about 15, about 20, about 25, about 30, or about 35 nucleotides in length. In some embodiments, the first and second probe-resolution barcode sequences can be about 3, 4, 5, 6, 7, 8, 9, 10, 11, 12, 13, 14, 15, 16, 17, 18, 19, 20, 21, 22, 23, 24, 25, or more nucleotides in length. In some embodiments, the first and second probe-resolution barcode sequences can be of the same length.

In some embodiments, the target-specific barcode sequence can be longer than the first and second probe-resolution barcode sequences. In some embodiments, the target-specific barcode sequence can be between about 15 and about 25 nucleotides in length, and the first and second probe-resolution barcode sequences can be between about 3 and about 10 nucleotides in length. In some embodiments, the target-specific barcode sequence can be about 20 nucleotides in length, and the first and second probe-resolution barcode sequences can be about 5 nucleotides in length.

In some embodiments, when the probes (or amplification products thereof, e.g., RCA products) associated with a plurality of different mRNA and/or cDNA analytes can be analyzed, a barcode sequence in a particular circular or circularizable (e.g., padlock) probe can uniquely correspond to a particular mRNA or cDNA analyte, and the particular circular or circularizable (e.g., padlock) probe can further comprise an anchor sequence that is common among circular or circularizable (e.g., padlock) probes for a subset of the plurality of different mRNA and/or cDNA analytes. In some embodiments, the first and/or second probes disclosed herein can further comprise an anchor sequence. In some embodiments, the anchor sequence or complement thereof in amplification products (e.g., RCA products) can be detected using detectable probes, e.g., an immediate probe (e.g., an L-shaped probe) that hybridizes to the anchor sequence or complement thereof and a fluorescently-labeled probe that hybridizes to immediate probe. Signals associated with the anchor sequence can be used to detect all amplification products (e.g., RCA products) that comprise the common anchor sequence or complement thereof. Thus, in some embodiments, signals associated with the anchor sequence can be used as controls during sequential cycles of detecting the target-specific barcode sequences and/or the probe-resolution barcode sequences (or complements thereof) in a plurality of amplification products (e.g., RCA products).

In some embodiments, the anchor sequence can be adjacent to the target-specific barcode sequence. In some embodiments, the anchor sequence can be separated from the 5′ or 3′ nucleotide of the target-specific barcode sequence by 0, 1, 2, 3, 4, 5, or more nucleotides. In some embodiments, the anchor sequence can be common between the first and second probes. In some embodiments, the anchor sequence can be common among the plurality of probes. In some embodiments, the anchor sequence can be common among probes targeting different nucleic acid molecules in the biological sample. In some embodiments, the anchor sequence can be about 5, about 10, about 15, about 20, about 25, about 30, or about 35 nucleotides in length. In some embodiments, the anchor sequence can be 5, 6, 7, 8, 9, 10, 11, 12, 13, 14, 15, 16, 17, 18, 19, 20, 21, 22, 23, 24, 25, 26, 27, 28, 29, 30 or more nucleotides in length. In some embodiments, the anchor sequence can be about 20 nucleotides in length. In some embodiments, the anchor sequence can be a linker sequence between a target-specific barcode sequence and a probe-resolution barcode sequence. In some embodiments, the anchor sequence can be comprised in or overlap with a linker sequence between a target-specific barcode sequence and a probe-resolution barcode sequence.

In some embodiments, the first and/or second probes can further comprise one or more linker sequences. In some embodiments, the first and/or second probes can comprise two linker sequences flanking the first or second probe-resolution barcode sequence, respectively. In some embodiments, each of the one or more linker sequences can be independently 1, 2, 3, 4, 5, 6, 7, 8, 9 or more nucleotides in length. In some embodiments, the one or more linker sequences can be common between the first and second probes. In some embodiments, the one or more linker sequences can be common among the plurality of probes. In some embodiments, the one or more linker sequences can be common among probes targeting different nucleic acid molecules in the biological sample. In some embodiments, the one or more linker sequences can be used as an anchor sequence. That is, the first and/or second probes do not need to have separate anchor and linker sequences. In some embodiments, the one or more linker sequences can be comprised in or overlap with an anchor sequence.

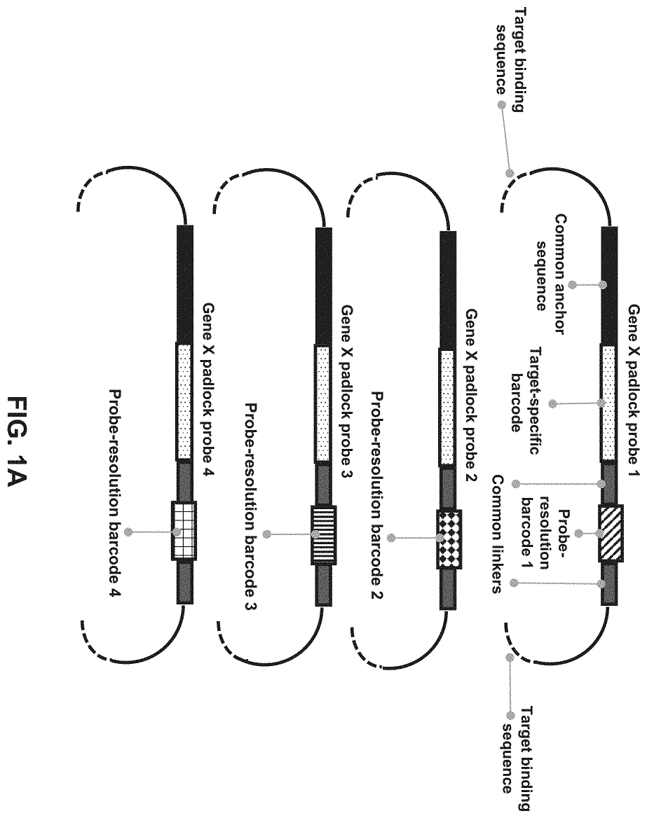

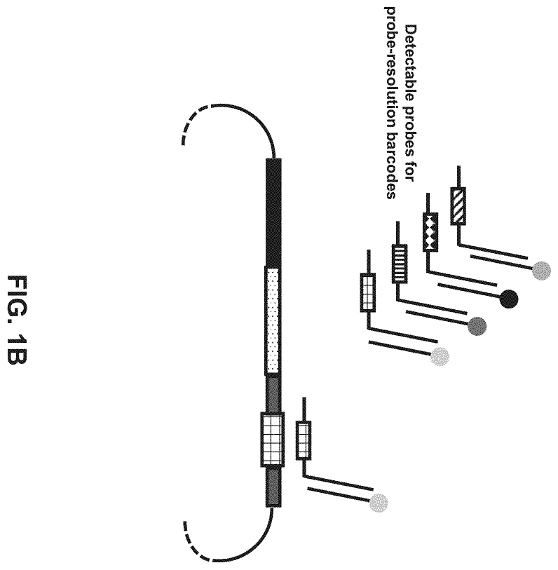

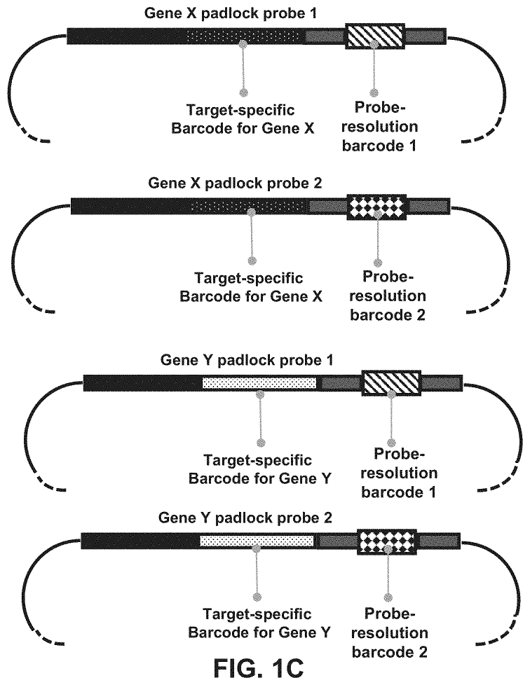

In some embodiments, a first probe targeting a target nucleic acid in a first species comprises a first target binding sequence complementary to a target sequence in the target nucleic acid in the first species, whereas a second probe targeting the same target nucleic acid (or a homolog thereof) in a second species comprises a second target binding sequence complementary to a target sequence in the target nucleic acid (or homolog thereof) in the second species. In some embodiments, the target binding sequences in the first and second probes can be species-specific, and the first and second probes can comprise the same target-specific barcode sequence corresponding to the target nucleic acid or homolog thereof but different species-specific barcode sequences corresponding to the first and second species, respectively. For instance, the target binding sequences in the four probes targeting Gene X shown in FIG. 1A can be different from each other and species-specific. Similarly, the target binding sequences in the two probes targeting Gene Y shown in FIG. 1C can be different from each other and species-specific.

In some embodiments, the first and/or second probe-resolution barcode sequences can be adjacent to the target-specific barcode sequence. In some embodiments, the first and/or second probe-resolution barcode sequences can be separated from the 5′ or 3′ nucleotide of the target-specific barcode sequence by 0, 1, 2, 3, 4, 5, or more nucleotides.

In some embodiments, the plurality of probes can further comprise a third probe comprising a third probe-resolution barcode sequence, and the method can further comprise detecting a signal associated with the third probe-resolution barcode sequence. In some embodiments, the plurality of probes can further comprise a fourth probe comprising a fourth probe-resolution barcode sequence, and the method can further comprise detecting a signal associated with the fourth probe-resolution barcode sequence. In some embodiments, the signals associated with different probe-resolution barcode sequences can be detected in separate detection channels, such as different fluorescent channels. As an example, detectable probes for the first, second, third, and fourth probe-resolution barcode sequences (or complements thereof) can be contacted with the biological sample all at once, and the signal associated with each probe-resolution barcode sequence can be detected in one of red, green, blue, and yellow fluorescent channels. In some embodiments, the first, second, third, and/or fourth probe-resolution barcode sequences can be different among probes targeting the same nucleic acid molecule. Thus, probes (e.g., padlock probes) targeting the same nucleic acid molecule can be divided into subsets based on the probe-resolution barcode sequence in each particular probe, and each subset can be detected in a separate detection channel, although in some embodiments, different subsets (due to different probe-resolution barcode sequences in the probes) may be detected in the same detection channel (e.g., simultaneously). For instance, probes (e.g., padlock probes) comprising the same target-specific barcode sequence and targeting the same analyte can be divided into five subsets, each subset of probes comprising a different probe-resolution barcode sequence. Amplification products (e.g., RCA products) of the five subsets of padlock probes can be detected using detectable probes (e.g., an L-shaped probe and a fluorescently labeled probe binding to the L-shaped probe) in five separate fluorescent channels, one channel for RCA products of each subset. Alternatively, amplification products (e.g., RCA products) of any two or more of the subsets can be detected in the same fluorescent channel. For example, RCA products of two subsets can be detected in red, while RCA products of the other three subsets can be detected in green, blue, and yellow, respectively. In some embodiments, one or more subsets of probes (e.g., padlock probes) are not detected. In other words, it is not necessary to detect each and every one of the different probe-resolution barcode sequences in the probes targeting a particular analyte. In some embodiments, the detection of a target-specific barcode sequence and the detection of one or more (but not all) different probe-resolution barcode sequences in probes for a particular analyte are sufficient to resolve all or a portion of overcrowding signals associated with the analyte. In such examples, the remaining probe-resolution barcode sequence(s) may but need not be detected.

In some embodiments, a biological sample may contain some highly-expressed or abundant targets, which may be analyzed by using the probe-resolution barcode sequences, while other targets resolvable with the target-specific barcode may not need the use of probe-resolution barcode sequences. In some embodiments, a biological sample may first be analyzed using the target-specific barcode sequences and if overlapping signals are detected, the sample may be further analyzed by using the probe-resolution barcode sequences.

In some embodiments, provided herein are methods and compositions for detecting the origins of cells and analytes in and/or on the cells in a biological sample. In some embodiments, the biological sample can be contacted with multiple circular or circularizable probes or probe sets that target a single gene (e.g., a genomic DNA, an RNA, or a cDNA), wherein each probe or probe set comprises a probe-resolution barcode sequence (“species-specific tag”) that corresponds to the species that the probe or probe set is designed for. In some embodiments, a species-specific probe-resolution barcode sequence disclosed herein does not specifically correspond to any particular target analyte(s) but can be used to identify the species origins of one or more target analytes. By labelling and detecting the species-specific probe-resolution barcode sequences using their corresponding detectable probes (e.g., L-shaped probes comprising hybridization regions for the probe-resolution barcode sequences or complements thereof and overhangs for hybridization and detection by fluorescently labelled probes), each gene can be detected in different and multiple fluorescent channels. In some cases, detecting of the signals across different channels allows identification of subsets of the signals associated with the same target analyte to be associated with a particular origin (e.g., species origin such as mouse or human). The same target analyte may include homologs of the same gene in different species, such as mouse Malat-1 and human MALAT-1, and probes targeting the same target analyte from different species may have the same or different target binding sequences (e.g., depending on how much sequence difference there is for the same gene in different species), the same target-specific barcode sequence (e.g., gene-specific barcode sequence corresponding to the same gene in different species), and different species-specific barcode sequences each corresponding to a species. In some embodiments, a first probe targeting a gene in a first species comprises a first target binding sequence complementary to a target sequence for the gene in the first species, whereas a second probe targeting the same gene or a homolog thereof in a second species comprises a second target binding sequence complementary to a target sequence for the gene or homolog in the second species. The first and second probes can comprise the same target-specific barcode sequence corresponding to the gene or homolog but different species-specific barcode sequences corresponding to the first and second species, respectively.

In some embodiments, a biological sample may contain target analytes of one or more origins (e.g., species origins), which may be analyzed by using the probe-resolution barcode sequences (e.g., species-specific tags), while other targets resolvable with the target-specific barcode may not need the use of probe-resolution barcode sequences. In some embodiments, a biological sample may be analyzed using the target-specific barcode sequences and once species origin of the cells are detected, other target analytes may be further analyzed without using the probe-resolution barcode sequences. The same probe-resolution barcode sequence (e.g., species-specific tag) can be used for multiple different target analytes of the same species. In some embodiments, a first plurality of probes may each comprise a first probe-resolution barcode sequence and each probe of the first plurality of probes may target various nucleic acid sequences (e.g., a plurality of target analytes) of a first species, and a second plurality of probes may each comprise a second probe-resolution barcode sequence and each probe of the second plurality of probes may target various nucleic acid sequences (e.g., a plurality of target analytes) of a second species.

In some embodiments, the first, second, third, and/or fourth probe-resolution barcode sequences can be common among probes targeting different nucleic acid molecules in the biological sample. For instance, a first pair of probes targeting Gene X and Gene Y respectively can share a common first probe-resolution barcode sequence, a second pair of probes targeting Gene X and Gene Y respectively can share a common second probe-resolution barcode sequence, a third pair of probes targeting Gene X and Gene Y respectively can share a common third probe-resolution barcode sequence, and a fourth pair of probes targeting Gene X and Gene Y respectively can share a common fourth probe-resolution barcode sequence. In some cases, the common probe-resolution barcode sequences shared among different targets can save on costs and reagents across target probe sets. In some embodiments, a set of probe-resolution barcode sequences is used per set of probes for each target. For example, four probe-resolution barcode sequences can be used in probes for a plurality of different targets, and each target may be targeted by four probes (e.g., padlock probes) each comprising one of the four probe-resolution barcode sequences.

In some embodiments, the plurality of probes can directly or indirectly bind to the same sequence in the nucleic acid molecule. In some embodiments, the first and second probes can hybridize to the same sequence in the nucleic acid molecule. In some embodiments, two or more of the plurality of probes can directly or indirectly bind to different sequences in the same nucleic acid molecule. In some embodiments, the first and second probes can hybridize to different sequences in the same nucleic acid molecule.

In some embodiments, the first and second probes can be circularizable probes or probe sets, for example, padlock probes including gap-filling padlock probes, SNAIL probes, molecular inversion probes, invader probes including invader padlock probes, and any of the probes or probe sets described in Section III. In some embodiments, the first and/or second probes can comprise a ribonucleotide, such as no more than four, no more than three, or no more than two ribonucleotides. In some embodiments, the first and second probes can be padlock probes, and ends of the padlock probes can be ligated using the nucleic acid molecule as a template, with or without gap filling prior to ligation. In some embodiments, the padlock probes can comprise deoxyribonucleotides and/or ribonucleotide(s), and the nucleic acid molecule can be an RNA molecule, such as mRNA. In some embodiments, the padlock probes can comprise a 3′ ribonucleotide in a deoxyribonucleotide backbone.

In some embodiments, a probe disclosed herein is amplified through rolling circle amplification. In some embodiments, the primary probes, such as a padlock probe or a probe set that comprises a padlock probe, contain one or more barcodes. In some embodiments, the barcodes are bound by detection primary probes, which do not need to be fluorescent, but that include a target-binding portion (e.g., for hybridizing to one or more primary probes) and multiple other barcodes (e.g., secondary barcodes, versus the primary barcodes on the primary probes). In some embodiments, the barcodes of the detection primary probes are targeted by detectably labeled detection oligonucleotides, such as fluorescently labeled oligos. In some embodiments, one or more decoding schemes are used to decode the signals, such as fluorescence, for sequence determination. Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, the method comprises using a circular or circularizable construct hybridized to the polynucleotides of interest to generate a circular nucleic acid. In some embodiments, the RCA comprises a linear RCA. In some embodiments, the RCA comprises a branched RCA. In some embodiments, the RCA comprises a dendritic RCA. In some embodiments, the RCA comprises any combination of the foregoing. In some embodiments, the circular nucleic acid is a construct formed using ligation. In some embodiments, the circular construct is formed using template primer extension followed by ligation. In some embodiments, the circular construct is formed by providing an insert between ends to be ligated. In some embodiments, the circular construct is formed using a combination of any of the foregoing. In some embodiments, the ligation is a DNA-DNA templated ligation. In some embodiments, the ligation is an RNA-RNA templated ligation. Exemplary RNA-templated ligation probes and methods are described in US 2020/0224244 which is incorporated herein by reference in its entirety. In some embodiments, the ligation is a RNA-DNA templated ligation. In some embodiments, a splint is provided as a template for ligation.

In some embodiments, a probe disclosed herein (e.g., a padlock probe) can comprise a 5′ flap which may be recognized by a structure-specific cleavage enzyme, e.g. an enzyme capable of recognizing the junction between single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to the target nucleic acid molecule, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiment, cleavage of the additional sequence 5′ to the first target-specific binding site is performed by a structure-specific cleavage enzyme, e.g. a Flap endonuclease. Suitable Flap endonucleases are described in Ma et al. 2000. JBC 275, 24693-24700 and in US 2020/0224244 and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g. dinucleotides or trinucleotides), or cleaving the entire 5′ single-stranded region, e.g. as described in Lyamichev et al. 1999. PNAS 96, 6143-6148 for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

Following formation the circular nucleic acid, in some instances, an amplification primer is added. In other instances, the amplification primer is added with the primary and/or secondary probes. In some instances, the amplification primer may also be complementary to the target nucleic acid and the padlock probe (e.g., a SNAIL probe). In some embodiments, a washing step is performed to remove any unbound probes, primers, etc. In some embodiments, the wash is a stringency wash. Washing steps can be performed at any point during the process to remove non-specifically bound probes, probes that have ligated, etc.

In some instances, upon addition of a DNA polymerase in the presence of appropriate dNTP precursors and other cofactors, the amplification primer is elongated by replication of multiple copies of the template. The amplification step can utilize isothermal amplification or non-isothermal amplification. In some embodiments, after the formation of the hybridization complex and any subsequent circularization (such as ligation of, e.g., a padlock probe) the circular probe is rolling-circle amplified to generate a DNA concatemer (e.g., amplicon) containing multiple copies of the circular.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

In some embodiments, rolling circle amplification products are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In some embodiments, the polymerase comprises a modified recombinant Phi29-type polymerase. In some embodiments, the polymerase comprises a modified recombinant Phi29, B103, GA-1, PZA, Phi15, BS32, M2Y, Nf, G1, Cp-1, PRD1, PZE, SFS, Cp-5, Cp-7, PR4, PR5, PR722, or L17 polymerase. In some embodiments, the polymerase comprises a modified recombinant DNA polymerase having at least one amino acid substitution or combination of substitutions as compared to a wildtype Phi29 polymerase. Exemplary polymerases are described in U.S. Pat. Nos. 8,257,954; 8,133,672; 8,343,746; 8,658,365; 8,921,086; and 9,279,155, all of which are herein incorporated by reference. In some embodiments, the polymerase is not directly or indirectly immobilized to a substrate, such as a bead or planar substrate (e.g., glass slide), prior to contacting a sample, although the sample may be immobilized on a substrate. In some embodiments, the polymerase is not attached to a nanopore, a nanopore membrane or an insulating support thereof.

Following amplification, the sequence of the amplicon or a portion thereof, is determined or otherwise analyzed, for example by using detectably labeled probes and imaging. The sequencing or analysis of the amplification products can comprise sequencing by hybridization, sequencing by ligation, and/or fluorescent in situ sequencing, and/or wherein the in situ hybridization comprises sequential fluorescent in situ hybridization. In some instances, sequencing using, e.g., the secondary and higher order probes and detection oligonucleotides described herein.

### V. Signal Amplification, Detection and Analysis

In some aspects, the provided methods involve analyzing, e.g., detecting or determining, one or more sequences present in the polynucleotides (e.g., probes described in Section III; probes comprising a target-specific barcode sequence and a probe-resolution barcode sequence as described in Section IV) and/or in a product or derivative thereof, such as in an amplification product (e.g., of an amplified padlock probe).

In some embodiments, the present disclosure addresses signal crowding in methods that involve detecting nucleic acid sequences (either as the target analytes or as the labels or reporters for one or more target analytes, such as one or more target proteins), including in situ assays that detect the localization of analytes in sample. There are a number of situations in which it is desired to detect several different analytes in a sample simultaneously, for example when detecting the expression of different genes in situ in a sample, where there can be a wide range of different expression levels. In some embodiments, nucleic acid molecules are detected as target analytes in situ in a sample. In some embodiments, nucleic acid molecules are detected as reporters for other, non-nucleic acid analytes, including for example proteins, or indeed as a reporter, or signal amplifier, for a nucleic acid analyte. Thus, in a detection assay for such an analyte, nucleic acid molecules may be used, for example as a tag or reporter for an antibody or other affinity-binder-based probe (e.g. in immunoPCR or immunoRCA), or generated, for example by ligation or extension in a proximity probe-based assay. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template PCR amplification, as described for example in Soderberg et al., Methods (2008), 45(3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Application Publication No. 2002/0051986, the entire contents of which are incorporated herein by reference. In some embodiments, two analytes in proximity can be specifically bound by two labelling agents (e.g., antibodies) each of which is attached to a reporter oligonucleotide (e.g., DNA) that can participate, when in proximity when bound to their respective targets, in ligation, replication, and/or sequence decoding reactions. The nucleic acid molecule may be present in an amount which reflects the level of the analyte and may be detected as a “proxy” for the target analyte. Suitable methods for detecting multiple nucleic acid sequences in a sample can include the use of hybridization probes and sequencing-by-hybridization.

In some embodiments, a method disclosed herein comprises labelling analytes to be detected (either directly or indirectly) with detectable labels, using hybridization probes for example, and then detecting signals from those labels in order to identify the nucleic acid sequences. In some embodiments, some of the target nucleic acid sequences are present in the sample at significantly higher or lower concentrations than the other target nucleic acid sequences. If a particular target nucleic acid sequence is present in the sample at a high concentration, then a large number of hybridization probes will be bound to that target nucleic acid sequence and a large number of signals will be generated. In some embodiments, multiple signals are generated and detected concurrently, and the number of signals that are generated from each target nucleic acid sequence is related to the amount of that target nucleic acid sequence which is present in the sample. Accordingly, signals from target nucleic acid sequences which are present in high concentrations or in close proximity to signals from other target nucleic acid sequences may overcrowd and mask signals from the target nucleic acid sequences. In some embodiments, a method disclosed herein prevents and/or ameliorates signal crowding in multiplex assays where it is desired to detect a number of different nucleotide sequences, regardless of the means by which the sequences are labelled, and the type of labelling that is used (e.g. optical signals, radioactive signals, etc.). The present disclosure is particularly useful where a number of different signals are being generated simultaneously in close proximity.

In some embodiments, a method disclosed herein comprises detecting and identifying RNA transcripts in a given cell, in order to analyze the gene expression of that cell. In some embodiments, a method disclosed herein comprises labelling the RNA transcripts (or one or more primary or higher order probes bound thereto) with fluorescently labelled probes. The signals from the fluorescent labels can then be visualized in order to determine which RNA transcripts are present in a given cell of, e.g., a tissue sample. This can also be used to provide information on the location and the relative quantities of different RNA transcripts (and therefore the location and relative levels of expression of the corresponding genes). If a particular gene (or genes) is significantly overexpressed, a large number of RNA transcripts corresponding to that gene will be present in the sample, and thus a large number of fluorescent signals indicating the presence of that RNA transcript will be generated. At a certain point, the signal density will be such that at least some individual signals cannot be resolved using conventional fluorescence microscopy, thereby inhibiting or even preventing the detection of signals from other RNA transcripts corresponding to genes which are expressed at a lower level or that physically overlap or are otherwise in close proximity in the sample (either in 2D or 3D space), which leads to a loss of information and an inaccurate picture of gene expression. It will be understood that this problem can occur in many other nucleic acid detection methods. In some aspects, the present disclosure provides a method of detecting multiple target nucleic acid sequences in a sample wherein signal crowding is reduced.

In some embodiments, the methods provided in this disclosure are for use in the multiplexed detection of analytes (such as nucleic acids), that is, for the detection of multiple target analytes in a sample, e.g., one or more tissue samples such as a single tissue section or a series of tissue sections. In some embodiments, the methods use hybridization probes, whilst reducing signal crowding from said hybridization probes. In some embodiments, the methods provided herein comprise sequencing-by-hybridization (SBH) or sequential hybridization of probes for detecting nucleic acid sequences in a sample, including multiplex SBH or sequential hybridization of probes for detecting different target nucleic acid sequences (e.g., labels or reporters for one or more target analytes), with a wide range of distribution and abundance simultaneously in a sample. In some embodiments, the methods provided herein address signal crowding issues due to signals indicative of target nucleic acid sequences present in high concentrations and/or close proximity that may mask and/or overcrowd other signals.

In some aspects, signal overcrowding may prevent signals relating to the target nucleic acid sequences from being generated, detected, or otherwise distinguished from other signals in the sample. For example, if the hybridization probes cannot successfully hybridize to their cognate target nucleic acid sequences due to steric hindrances, or if detection probes cannot hybridize to the hybridization probes, then signals will not be generated and thus the target nucleic acid sequences will not be detected. This may be referred to as steric crowding. Alternatively, it may be that signals are properly generated from all of the target nucleic acid sequences, but that so many signals are generated, either in a particular area of the sample or in the sample as a whole (e.g., the signal density is too large), that not all of the signals can be properly detected and resolved. Where the signals are detected by optical means, this may be referred to as optical crowding, and the present methods are particularly suited to resolving, or reducing, optical crowding. In some aspects, by “optical means” is meant that the signals are detected visually, or by visual means, namely that the signals are visualized. Thus, in some instances, the signals that are generated involve detection of light or other visually detectable electromagnetic radiation (such as fluorescence). In some aspects, the signals may be optical signals, visual signals, or visually detectable signals. The signals may be detected by sight, typically after magnification, but more typically they are detected and analyzed in an automated system for the detection of the signals.

In some aspects, the signals may be detected by microscopy. In some aspects, an image may be generated in which the signals may be seen and detected, for example an image of the field of view of a microscope, or an image obtained from a camera. The signals may be detected by imaging, more particularly by imaging the sample or a part or reaction mixture thereof. By way of example, signals in an image may be detected as “spots” which can be seen in the image. In some aspects, a signal may be seen as a spot in an image. In some aspects, optical crowding occurs when individual spots cannot be resolved, or distinguished from one another, for example when they overlap, or mask one another. By reducing the number of spots using the methods herein, such that individual spots, or signals, can be resolved, optical crowding can be reduced. In some aspects, the present methods optically de-crowd the signals.

In some aspects, the methods herein involve reducing the number of signals that are detected at once in a detection step of the method. This is achieved in different ways in the different methods, to prevent or block a signal from being generated from certain targets (e.g., abundant or highly expressed targets in a sample) in a given cycle of detection. The targets may include highly expressed genes (e.g., mRNA transcripts) or abundant molecules that are targeted by labelling agents (e.g., reporter oligonucleotide-conjugated antibodies).

In some embodiments, a method disclosed herein may also comprise one or more signal amplification components. In some embodiments, the present disclosure relates to the detection of nucleic acids sequences (e.g., target-specific barcode sequences and/or probe-resolution barcode sequences) in situ using probe hybridization and generation of amplified signals associated with the probes, wherein background signal is reduced and sensitivity is increased.

In some embodiments, a barcode sequence (e.g., the target-specific barcode sequences, the probe-resolution barcode sequences, or the species-specific barcode sequences) can be in a rolling circle amplification (RCA) product molecule, a complex comprising an initiator and an amplifier for hybridization chain reaction (HCR), a complex comprising an initiator and an amplifier for linear oligonucleotide hybridization chain reaction (LO-HCR), a primer exchange reaction (PER) product molecule, a complex comprising a pre-amplifier and an amplifier for branched DNA (bDNA), or a complex comprising any two or more of the aforementioned molecules and complexes. For example, a bDNA complex or an HCR complex can be assembled on an RCA product. See, e.g., US 2021/0198727, incorporated herein by reference in its entirety.

A signal associated with a probe disclosed herein (e.g., signals associated with the target-specific barcode sequences, the probe-resolution barcode sequences, or the species-specific barcode sequences) can be detected using a method comprising targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER).

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, US 2022/0026433, US 2022/0128565, and US 2021/0222234, all of which are incorporated herein by reference in their entireties.

In some embodiments, a signal associated with a probe disclosed herein (e.g., signals associated with the target-specific barcode sequences, the probe-resolution barcode sequences, or the species-specific barcode sequences) can be detected using hybridization chain reaction (HCR). HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. No. 7,632,641 and U.S. Pat. No. 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. For exemplary complexes, see e.g., US 2020/0399689 and US 2022/0064697, which are fully incorporated by reference herein.

In some embodiments, a signal associated with a probe disclosed herein (e.g., signals associated with the target-specific barcode sequences, the probe-resolution barcode sequences, or the species-specific barcode sequences) can be detected using linear oligo hybridization chain reaction (LO-HCR). In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be a sequence of an endogenous analyte or RCA product. Exemplary methods and compositions for LO-HCR are described in US 2021/0198723, incorporated herein by reference in its entirety.

In some embodiments, the barcode sequences (e.g., of a probe or RCA product comprising target-specific and/or probe-resolution barcode sequences as described in Section IV) can be detected in with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising probes or RCA products (e.g., comprising target-specific and/or probe-resolution barcode sequences as described in Section IV) generated using methods described herein. In various embodiments, the probes or RCA products (e.g., comprising target-specific and/or probe-resolution barcode sequences as described in Section IV) may be contacted with a plurality of concatemer primers and a plurality of labeled probes. See e.g., U.S. Pat. Pub. No. US2019/0106733, which is incorporated herein by reference, for exemplary molecules and PER reaction components.

In some embodiments, the probes or RCA products (e.g., comprising target-specific and/or probe-resolution barcode sequences as described in Section IV) can be detected by providing detection probes, such as probes for performing a chain reaction that forms an amplification product, e.g., HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the polynucleotide probes (e.g., a barcode sequence present in an overhang region of the first and/or second probe).

In some embodiments, the methods comprise sequencing all or a portion of the amplification product, such as one or more barcode sequences (e.g., target-specific and/or probe-resolution barcode sequences as described in Section IV) present in the amplification product. In some embodiments, the analysis and/or sequence determination comprises sequencing all or a portion of the amplification product or the probe(s) and/or in situ hybridization to the amplification product or the probe(s). In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is incorporated herein by reference, for exemplary probes and HCR reaction components. In some embodiments, the detection or determination comprises hybridizing to the amplification product a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some aspects, the provided methods comprise imaging the amplification product (e.g., amplicon) and/or one or more portions of the polynucleotides, for example, via binding of the detection probe and detecting the detectable label. In some embodiments, the detection probe comprises a detectable label that can be measured and quantitated. The terms “label” and “detectable label” comprise a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, Ypet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenicol acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more polynucleotide(s) and/or amplification products (e.g., amplicon) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2^{nd} Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores can include those described in Henegariu et al. (2000) Nature Biotechnol. 18:345, incorporated herein by reference.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932. Exemplary techniques for in situ sequencing comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121).

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232.

In some embodiments, sequence analysis of nucleic acids can be performed by sequential hybridization (e.g., sequencing by hybridization and/or sequential in situ fluorescence hybridization). Sequential fluorescence hybridization can involve sequential hybridization of detection probes comprising an oligonucleotide and a detectable label. In some embodiments, a method disclosed herein comprises sequential hybridization of the detectable probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectable probes are described in US 2019/0161796, US 2020/0224244, US 2022/0010358, US 2021/0340618, and WO 2021/138676, all of which are incorporated herein by reference.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597.

In some embodiments, the barcodes of the probes (e.g., the padlock probe or the first and/or second probe) are targeted by detectably labeled detection oligonucleotides, such as fluorescently labeled oligonucleotides. In some embodiments, one or more decoding schemes are used to decode the signals, such as fluorescence, for sequence determination. In some embodiments, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some cases, the analysis is performed on one or more images captured, and may comprise processing the image(s) and/or quantifying signals observed. In some embodiments, images of signals from target-specific barcode detection in one fluorescent channel and the probe-resolution barcode detection in separate fluorescent channels can be compared and analyzed. In some embodiments, images of signals from target-specific barcode detection in one fluorescent channel and the probe-resolution barcode detection in separate fluorescent channels can be aligned to resolve individual signals. For example, the analysis may comprise processing information of one or more cell types, one or more types of biomarkers, a number or level of a biomarker, and/or a number or level of cells detected in a particular region of the sample. In some embodiments, the analysis comprises detecting a sequence e.g., a barcode present in the sample. In some embodiments, the analysis includes quantification of puncta (e.g., if amplification products are detected). In some cases, the analysis includes determining whether particular cells and/or signals are present that correlate with one or more biomarkers from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

In some embodiments, described herein is a method of localized detection of multiple target nucleic acids in a sample, wherein each target nucleic acid is targeted by a circular or circularizable primary probe specific for said target nucleic acid, and the circularizable primary probe can be circularized upon hybridization to the target nucleic acid. Each of a plurality of circular or circularized probes can comprise a target-specific barcode sequence corresponding to the target nucleic acid, and the plurality of circular or circularized probes can comprise different subsets of probes comprising different probe-resolution barcode sequences. The plurality of circular or circularized probes can bind to different molecules of the target nucleic acid at multiple locations in the sample, and can be amplified in situ by rolling circle amplification (RCA) to produce a rolling circle product (RCP). Each RCP can comprise multiple complementary copies of the target-specific barcode sequence and one of the different probe-resolution (e.g., species-specific) barcode sequences, wherein the target-specific barcode sequence and/or the probe-resolution barcode sequences can be decoded in multiple sequential decoding cycles each using hybridization probes (e.g., intermediate probes such as L-shaped probes) which hybridize to the complementary copies of the barcode sequences in an RCP and allow detectable signals to be generated. Signals associated with the target-specific barcode sequence in the sequential decoding cycles together yield a signal code sequence which can be used to identify the target-specific barcode sequence and its corresponding target nucleic acid sequence. Likewise, signals associated with the probe-resolution (e.g., species-specific) barcode sequences in the sequential decoding cycles can yield signal code sequences which can be used to identify the probe-resolution (e.g., species-specific) barcode sequences.

In some embodiments, provided herein is a method of analyzing a sample, comprising: a) producing an amplification product such as RCA product in the sample, the amplification product comprising multiple copies of a target-specific barcode sequence and one of a plurality of different probe-resolution barcode sequences, wherein the target-specific barcode sequence is associated with a target analyte and is assigned a signal code sequence, and wherein the sample is a cell or tissue sample; b) contacting the sample with a first intermediate probe and a first detectable probe to generate a first complex comprising the first intermediate probe hybridized to the amplification product and the first detectable probe hybridized to the first intermediate probe, wherein the first intermediate probe comprises (i) a hybridization region complementary to the target-specific barcode sequence and (ii) a first overhang sequence, and wherein the first detectable probe comprises (i) a sequence complementary to the first overhang sequence and (ii) a first optically detectable moiety; c) imaging the sample to detect a first signal from the first optically detectable moiety, wherein the first signal corresponds to a first signal code in the signal code sequence; d) contacting the sample with a second intermediate probe and a second detectable probe to generate a second complex comprising the second intermediate probe hybridized to the amplification product and the second detectable probe hybridized to the second intermediate probe, wherein the second intermediate probe comprises (i) a hybridization region complementary to the target-specific barcode sequence and (ii) a second overhang sequence, and wherein the second detectable probe comprises (i) a sequence complementary to the second overhang sequence and (ii) a second optically detectable moiety; and e) imaging the sample to detect a second signal from the second optically detectable moiety, wherein the second signal corresponds to a second signal code in the signal code sequence, wherein the signal code sequence comprising at least the first signal code and the second signal code is determined at a location in the sample, thereby decoding the target-specific barcode sequence and identifying the target analyte at the location in the sample. In some embodiments, the target-specific barcode sequence barcode sequence associated with the target analyte is selected from a plurality of barcode sequences, wherein the method comprises contacting the sample with a first pool of intermediate probes and a universal pool of detectable probes, wherein the first pool of intermediate probes comprises the first intermediate probe and the universal pool of detectable probes comprises the first detectable probe and the second detectable probe, wherein each intermediate probe in the first pool of intermediate probes comprises (i) a hybridization region complementary to one of the plurality of the target-specific barcode sequences and (ii) an overhang sequence complementary to a detectable probe of the universal pool of detectable probes; and the method comprises contacting the sample with a second pool of intermediate probes and the universal pool of detectable probes, wherein the second pool of intermediate probes comprises the second intermediate probe, and wherein each intermediate probe in the second pool of intermediate probes comprises (i) a hybridization region complementary to one of the plurality of the target-specific barcode sequences and (ii) an overhang sequence complementary to a detectable probe of the universal pool of detectable probes. In some embodiments, the method comprises identifying multiple different target analytes present at locations in the sample, wherein each different target analyte is assigned a different signal code sequence and is targeted by a circularizable probe or probe set comprising a complement of a different target-specific barcode sequence of the plurality of target-specific barcode sequences. In some embodiments, the number of different intermediate probes in each pool of intermediate probes is greater than the number of different detectable probes in the universal pool of detectable probes. In some embodiments, the number of different detectable probes in the universal pool of detectable probes is four. In some embodiments, the number of different intermediate probes in each pool of intermediate probes is about 10, about 20, about 50, about 100, about 200, about 500, about 1,000, about 2,000, about 5,000, or more.

In some embodiments, each probe-resolution barcode sequence or species-specific barcode sequence disclosed herein can be detected using sequential hybridization of intermediate probes (e.g., L-shaped probes) and detectable probes (e.g., fluorescently labeled probes) as described for the detection of target-specific barcode sequences herein. FIG. 1B shows a probe-resolution barcode sequence can be detected using sequential hybridization of intermediate probes comprising overhangs that hybridize to fluorescently labeled probes. The overhangs of the intermediate probes can mediate and/or initiate signal enhancement or amplification, such as hybridization chain reaction (HCR), linear oligonucleotide hybridization chain reaction (LO-HCR), or primer exchange reaction (PER), or any other signal enhancement or amplification methods described herein.

Signals associated with the probe-resolution barcode sequences can be used to facilitate registration of signals detected in the sequential cycles for decoding. In some embodiments, different subsets of amplification products associated with the same gene can be detected in different fluorescent channels, for example by detecting a probe-resolution barcode sequence of a first probe in a first fluorescent channel and detecting a different probe-resolution barcode sequence of a second probe in another fluorescent channel. The different subsets of probe-resolution barcode sequences can be detected separately (e.g., in different “color” channels), thereby alleviating signal crowding due to overlapping of signals associated with the same target-specific barcode sequence.

In some embodiments, a sequential decoding scheme involves detecting repeated signals from a given target in multiple cycles, and the target may be in the same position in the sample in the different cycles. In some embodiments, a method disclosed herein comprises the localized detection of the target nucleic acid sequences. In some embodiments, the target nucleic acid sequence is present at a fixed or defined location in the sample, and is detected at that location. The target nucleic acid sequence may be localized by virtue of being present in situ at its native location in the sample (e.g. a cell or tissue sample), or of being attached or otherwise localized to a target analyte which is present in situ at its native location in the sample. The target nucleic acid sequence may be immobilized in the sample, e.g., via crosslinking to other molecules in the sample or a matrix embedding the sample.

In some embodiments, image registration is performed. In some aspects, image registration comprises aligning signals and/or images obtained from various cycles onto a common coordinate system. When obtaining images or detecting signals from a sample across multiple cycles, the sample or imaging apparatus may shift, causing an offset of images from one cycle to the next. In some aspects, image registration compensates for these shifts, allowing the user to identify the same relative location within the sample between different images, and/or overlay images that are spatially aligned. In some embodiments, signals associated with the probe-resolution barcode sequences are used for image registration. In some embodiments, signals associated with each individual probe-resolution barcode sequence can provide a plurality of physical landmarks within the sample that can be used to align multiple images. In some embodiments, image registration allows decoding signals from multiple cycles to be assigned to the same location, allowing a signal code sequence to be constructed for that location. In some embodiments, image registration is performed using computational methods. In some embodiments, image registration is performed manually, guided, or adjusted by a user.

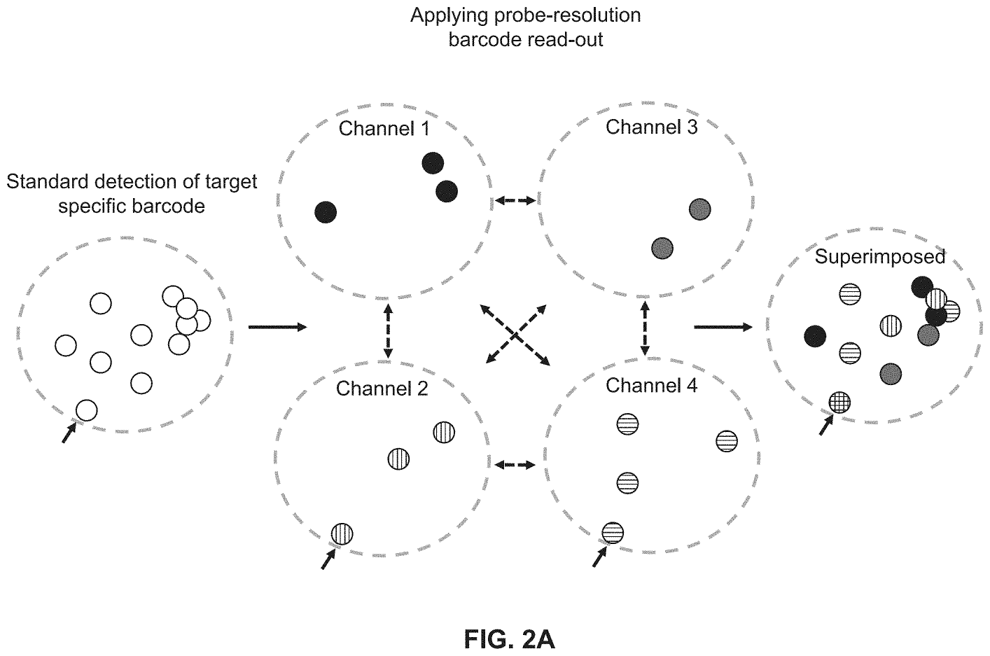

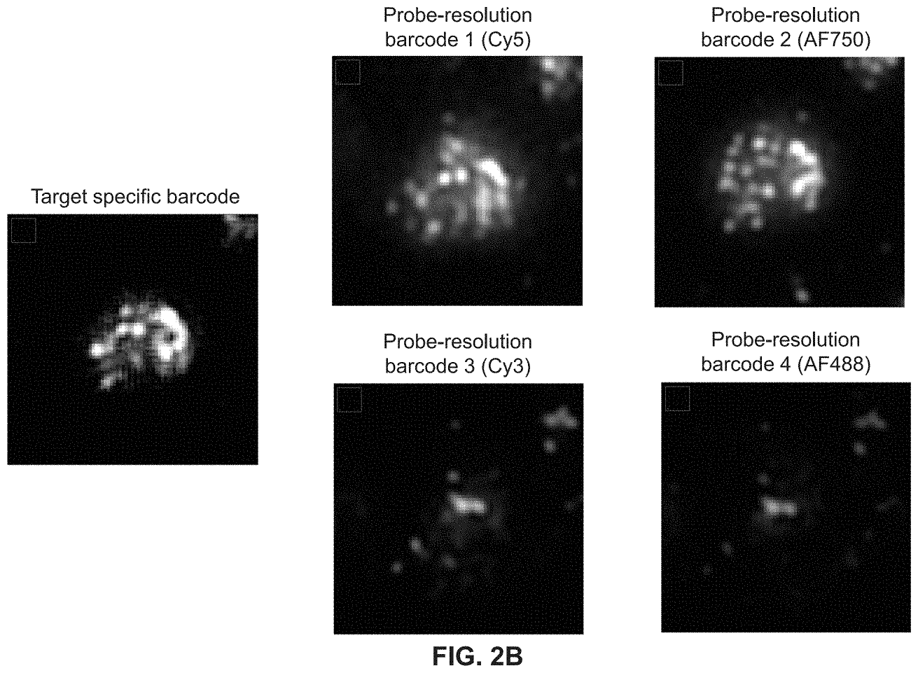

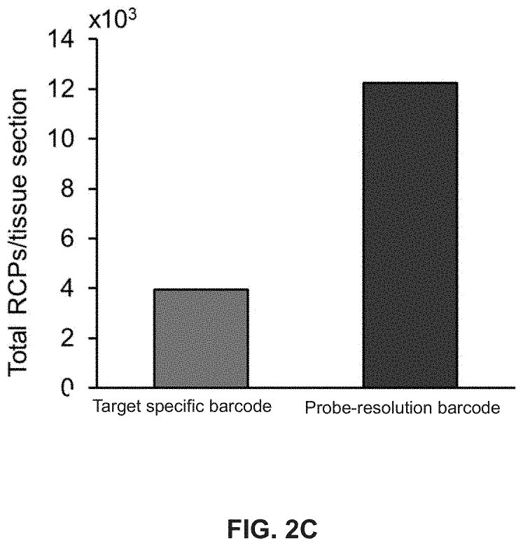

FIG. 2A illustrates that signals are initially detected with detectable probes for a target-specific barcode sequence corresponding to a target analyte, where some signals are overlapping and cause optical crowding. Some signals are partially overlapping, whereas other signals (e.g., the one indicated by the arrow) can be completely overlapping. By detecting signals associated with the probe-resolution barcode sequences, signals associated with the same target analyte can be detected in different color channels (e.g., Channels 1-4 such as Cy5, AF750, Cy3, and AF488 shown in FIG. 2B). In some cases, the signals detected in each of the different color channels are not overlapping and can be spatially resolved. Using image registration, signals associated with the probe-resolution barcode sequences can be associated with the signals associated with the target-specific barcode sequence, thereby resolving the overlapping signals (e.g., partially or completely overlapping signals).

### VI. Kits

Also provided herein are kits, for example comprising one or more polynucleotides, e.g., any described in Sections III and IV comprising target-specific and/or probe-resolution barcode sequences, and reagents for performing the methods provided herein, for example reagents required for one or more steps comprising hybridization, ligation, amplification, detection, sequencing, and/or sample preparation as described herein. In some embodiments, the kit further comprises a target nucleic acid, e.g., any described in Sections III and IV. In some embodiments, any or all of the polynucleotides are DNA molecules. In some embodiments, the target nucleic acid is a messenger RNA molecule. In some embodiments, the kit further comprises a ligase, for instance for forming a circular probe from the padlock probe. In some embodiments, the ligase has DNA-splinted DNA ligase activity. In some embodiments, the ligase has RNA-splinted ligase activity. In some embodiments, the kit further comprises a polymerase, for instance for performing amplification of the padlock probe, e.g., using any of the methods described in Section V. In some embodiments, the kit further comprises a primer for amplification.

In some embodiments, disclosed herein is a kit for analyzing a biological sample, comprising a plurality of probes each comprising a target-specific barcode sequence, e.g., a barcode sequence that corresponds to a target, such as a nucleic acid analyte or a protein analyte. In some embodiments, the plurality of probes comprise a first probe comprising a first probe-resolution barcode sequence and a second probe comprising a second probe-resolution barcode sequence different from the first probe-resolution barcode sequence. In some embodiments, the kit comprises a first plurality of probes comprising a first probe-resolution barcode sequence that target analytes (e.g., nucleic acid sequences) of a first species and a second plurality of probes comprising a second probe-resolution barcode sequence that target analytes (e.g., nucleic acid sequences) of a second species. In some embodiments, the plurality of probes target a nucleic acid molecule in the biological sample, such as a nucleic acid analyte (e.g., genomic DNA, mtDNA, cellular RNA such as mRNA, miRNA, etc., cDNA, or a product of a cellular nucleic acid) or a reporter oligonucleotide of a labelling agent (e.g., a nucleic acid tag conjugated to an antibody to a protein of interest). In some embodiments, the target-specific barcode sequence corresponds to the nucleic acid molecule. In some embodiments, the kit further comprises detectable probes that directly or indirectly bind to the target-specific barcode sequence or complement thereof. In some embodiments, the kit further comprises detectable probes that directly or indirectly bind to the first probe-resolution barcode sequence or complement thereof. In some embodiments, the kit further comprises detectable probes that directly or indirectly bind to the second probe-resolution barcode sequence or complement thereof.

In some embodiments, disclosed herein is a kit for analyzing a biological sample, comprising a plurality of padlock probes comprising a first padlock probe and a second padlock probe, wherein the first padlock probe comprises a target-specific barcode sequence and a first probe-resolution barcode sequence, and a second padlock probe comprises the target-specific barcode sequence and a second probe-resolution barcode sequence, and wherein the plurality of padlock probes hybridize to different nucleic acid molecules in the biological sample, and the target-specific barcode sequence corresponds to a particular nucleic acid molecule. In some embodiments, the kit further comprises a first intermediate probe that hybridizes to the complement of the target-specific barcode sequence and a first fluorescently labeled probe that hybridizes to the first intermediate probe. In some embodiments, the kit further comprises a second intermediate probe that hybridizes to the complement of the first probe-resolution barcode sequence and a second fluorescently labeled probe that hybridizes to the second intermediate probe. In some embodiments, the kit further comprises a third intermediate probe that hybridizes to the complement of the second probe-resolution barcode sequence and a third fluorescently labeled probe that hybridizes to the third intermediate probe. In some embodiments, the second and third fluorescently labeled probes are detectable in different fluorescent channels. In some embodiments, the first fluorescently labeled probe is detectable in the same or different fluorescent channels as the second fluorescently labeled probe or the third fluorescently labeled probe.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### VII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect regions of interest in target nucleic acids.

In some embodiments, the region of interest comprises a single-nucleotide polymorphism (SNP). In some embodiments, the region of interest comprises is a single-nucleotide variant (SNV). In some embodiments, the region of interest comprises a single-nucleotide substitution. In some embodiments, the region of interest comprises a point mutation. In some embodiments, the region of interest comprises a single-nucleotide insertion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G).

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

Two nucleic acid sequences may become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

### (ix) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments (e.g., when the PCR amplification amplifies captured DNA), the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oN™ DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as Rnase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)₂ fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay, a capture probe or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labelled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, capture probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine 0-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, Mito Tracker® Green, Mito Tracker® Orange, Mito Tracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™1/PO-PRO™-1, POPO™3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families can provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLES

The following examples are included for illustrative purposes only and are not intended to limit the scope of the present disclosure.

### Example 1: Increasing the Dynamic Range of a Highly Expressed Gene in Mouse Brain Tissue Sections

When preparing nucleic acid libraries for in situ detection of highly expressed genes, the dynamic range can be hindered by optical crowding of signals associated with nucleic acid probes. This can result from many locally amplified probes in close proximity, impeding the precise quantification of the expression levels of highly expressed genes. This example shows how, by using the probe-resolution barcode strategy (high-resolution tag), this limitation can be overcome, achieving a higher dynamic range by splitting the signal of an individual highly expressed gene into signals that can be detected at different times, e.g., in different fluorescence channels.

### Methods

### Padlock Probe Targeting and RCA In Situ on Fixed Mouse Brain Tissue Sections:

Fresh frozen mouse brain samples were cryo-sectioned at 10 μm and collected on ThermoFisher Superfrost glass slides. Slides were left to thaw at room temperature and fixed with PFA in PBS. The slides were then washed in PBS to ensure PFA removal before permeabilization. The slides were then washed twice in PBS before dehydrating with an ethanol series. The slides were subsequently air dried before applying a Secure Seal chamber (Grace Bio-Labs) to each section.

For probe hybridization, four Malat-1 padlock probes targeting different sequences of the Malat-1 transcript were added to each tissue section in a secure seal chamber and incubated. Each of the padlock probes contained the same common Malat-1-specific barcode sequence and one of four probe-resolution barcode sequences. After probe incubation, the samples were then washed. For padlock probe ligation, a T4 RNA ligase and RNAse inhibitor were mixed and added into each secure seal chamber and incubated. Samples were then washed twice with PBS-T. For probe amplification, Phi29 was added to each tissue section in a secure seal chamber. Samples were incubated and then washed in PBS-T, after which the samples were ready for the in situ sequencing by hybridization (SBH) reaction. The resulting amplification products contained complementary sequences of the target-specific barcode sequence (e.g., Malat-1 gene-specific barcode sequence) and complementary sequences of the probe-resolution barcode sequences from the padlock probes used as template.

Labelling with Gene Specific Barcode:

Individual gene labelling was performed by providing a SBH mix containing SSC, formamide and SBH-gene specific oligonucleotides (e.g., L-shaped probes that hybridize to the complementary sequences of the target-specific barcode sequences in RCA products, each L-shaped probe with an overhang that hybridizes to a fluorescently-labeled probe). The reaction was incubated, the mix was then removed, and the tissue sections were washed in PBS-T. Sections were then incubated with a detection mix containing SSC, formamide and SBH-detection oligonucleotides (e.g., fluorescently-labeled probes that hybridize to the overhangs of the L-shaped probes bound to the complementary sequences of the target-specific barcode sequences in RCA products). The mix was removed, and the tissue sections were washed twice in PBS-T and washed with ethanol. The slides were left to dry and mounted with mounting medium and a cover slip and imaged using 20× objective Nikon microscope (Eclipse Ti2).

Labelling with Probe-Resolution Barcode:

The slide was immersed in PBS to remove cover slip and mounting media. Tissue sections were washed and then incubated with a hybridization mix containing SSC, formamide and High Resolution SBH probes (e.g., L-shaped probes that hybridize to the complementary sequences of the probe-resolution barcode sequences in RCA products, each L-shaped probe with an overhang that hybridizes to a fluorescently-labeled probe). The mix was then removed, and the tissue sections were washed in PBS-T. Sections were then incubated with a detection mix containing SSC, formamide and SBH-detection oligonucleotides (e.g., fluorescently-labeled probes that hybridize to the overhangs of the L-shaped probes bound to the complementary sequences of the probe-resolution barcode sequences in RCA products). The mix was removed, and the tissue sections were washed in PBS-T and then ethanol. The slides were left to dry and mounted with mounting medium and a cover slip and imaged using 20× objective Nikon microscope (Eclipse Ti2).

### Results

Detecting Malat-1, a highly expressed non-coding RNA, with padlock probes and RCA can result in optical crowding. Therefore, the precise quantification of the expression level of this gene is limited since it is not possible to resolve and quantify single amplified molecules. FIGS. 2A-2C show an illustration of the probe-resolution barcode (high-resolution tag) strategy: when detected with only probes for the target-specific barcodes, signal crowding occurs (left panel); when detected with probes for the additional probe-resolution barcodes besides the probes for the target-specific barcodes, the signals can be detect in different color channels and higher resolution can be achieved (FIG. 2A). In this example, the detection of Malat-1 in a mouse brain tissue section is shown as an example (FIG. 2B). The left panel of FIG. 2B shows fluorescence image of a representative cell in the tissue section using probes for the target-specific barcode sequences in a single channel. Some of the individual signals were not resolved due to their local proximity. The middle and right panels of FIG. 2B show fluorescence image of the same representative cell in the tissue section detected using probes for the probe-resolution barcode sequences, each detected in one of four different color channels: Cy5, AF750, Cy3, and AF488. Using this approach, about three times more RCPs in total were resolved and quantified (FIG. 2C, right bar) compared to using probes for the target-specific barcode sequences alone (FIG. 2C, left bar), thus increasing the dynamic range for detection involving highly expressed genes such as Malat-1

### Example 2: Detecting Species Origin in a Patient-Derived Xenograft (PDX) Mouse Model

Fresh frozen cryo-sectioned samples from a PDX mouse model of Diffuse Intrinsic Pontine Glioma (DIPG) were collected on glass slides. Slides were washed in PBS, permeabilized, washed twice in PBS before dehydrating with an ethanol series. The slides were subsequently air dried before applying a Secure Seal chamber (Grace Bio-Labs).

For probe hybridization, four MALAT-1 padlock probes targeting the human MALAT-1 transcript and four Malat-1 padlock probes targeting the mouse Malat-1 transcript were added to each tissue section in a secure seal chamber and incubated. Padlock probes targeting RPLP0 was also added to the sample. Each of the padlock probes contained the same common barcode sequence (corresponding to both human MALAT-1 and mouse Malat-1) and either a probe-resolution (species-specific) barcode sequence corresponding to human species (for probes targeting human MALAT-1 transcript) or a probe-resolution (species-specific) barcode sequence corresponding to mouse species (for probes targeting mouse Malat-1 transcript). After probe incubation, the samples were then washed. For padlock probe ligation, a T4 RNA ligase and RNAse inhibitor were mixed and added into each secure seal chamber and incubated. Samples were then washed twice with PBS-T. For probe amplification, Phi29 was added to each tissue section in a secure seal chamber. Samples were incubated and then washed in PBS-T, after which the samples were ready for the detection of the barcode sequences by hybridizing fluorescently-labeled probes. The resulting amplification products contained complementary sequences of the target-specific (e.g., gene-specific) barcode sequences and complementary sequences of the probe-resolution (species-specific) barcode sequences from the padlock probes used as template.

Individual gene labelling was performed substantially as described in Example 1 and imaged. The slides were immersed in PBS to remove cover slip and mounting media. Tissue sections were washed and then incubated with a hybridization mix containing SSC, formamide and probe-resolution (Cy5 for mouse species-specific barcode and Cy7 for human species-specific barcode) SBH probes (e.g., L-shaped probes that hybridize to the complementary sequences of the barcode sequences in RCA products corresponding to either human or mouse species, each L-shaped probe with an overhang that hybridizes to a fluorescently-labeled probe). The mix was then removed, and the tissue sections were washed in PBS-T. Sections were then incubated with a detection mix containing SSC, formamide and SBH-detection oligonucleotides (e.g., fluorescently-labeled probes that hybridize to the overhangs of the L-shaped probes bound to the complementary sequences of the probe-resolution barcode sequences in RCA products). The mix was removed, and the tissue sections were washed in PBS-T and then ethanol. The slides were left to dry, mounted, and imaged.

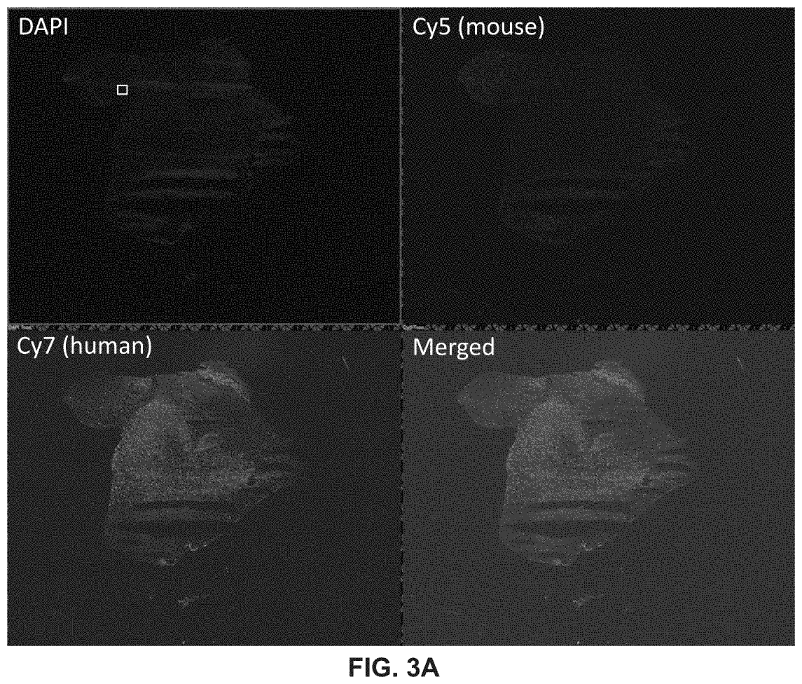

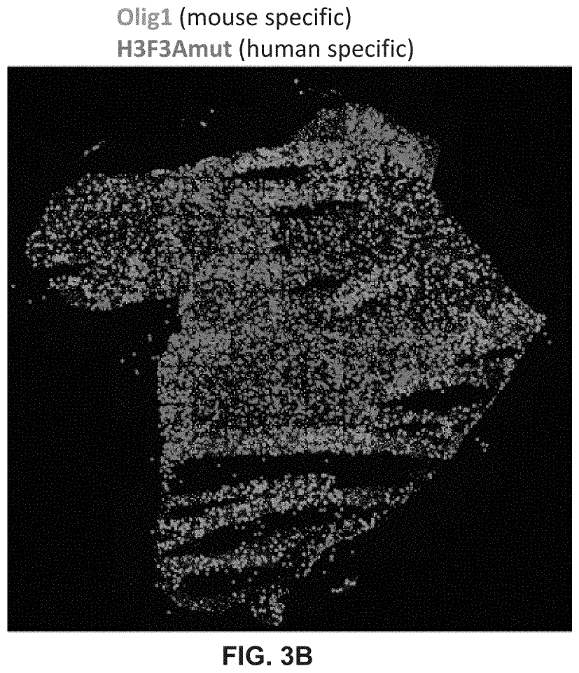

FIG. 3A shows fluorescence images of representative images of a tissue section detected in three different color channels: DAPI, Cy5, Cy7, and a merged image. To establish ground truth, on a consecutive tissue section, a cDNA-targeting padlock probe for targeting a human transcript that detects human specific H3F3A mutation (H3F3Amut) and a cDNA-targeting padlock probe for targeting a mouse transcript that detects a mouse specific Olig1 were used (e.g., performed following the method published by Ke et al., “In situ sequencing for RNA analysis in preserved tissue and cells,” (2013) Nature Methods 10:857-860). FIG. 3B shows overlaid images showing detection of the mouse specific barcode (corresponding to Olig1) and human specific barcode (corresponding to H3F3Amut). The patterns of human MALAT-1 (Cy7) and mouse Malat-1 (Cy5) expression detected in situ using probes for species-specific barcode sequences in FIG. 3A were consistent with the expression patterns of the human specific H3F3Amut and the mouse specific Olig1 detected in FIG. 3B. Using this approach, the species origin of cells in the PDX tissue sample was identified.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
(a) contacting the biological sample with a plurality of probes each comprising a target-specific barcode sequence,
wherein a first probe of the plurality of probes comprises a first probe-resolution barcode sequence and a second probe of the plurality of probes comprises a second probe-resolution barcode sequence, and
wherein the first probe targets a first molecule of a target analyte and the second probe targets a second molecule of the target analyte in the biological sample, and the target-specific barcode sequence corresponds to the target analyte;
(b) detecting a plurality of signals associated with the target-specific barcode sequences of the plurality of probes;
(c1) detecting a signal associated with the first probe-resolution barcode sequence; and
(c2) detecting a signal associated with the second probe-resolution barcode sequence,
wherein the signals of steps (c1) and (c2) are associated with the target analyte.

2. The method of claim 1, wherein the plurality of signals detected in step (b) comprise overlapping signals that are not spatially resolved into individual puncta in step (b).

3. The method of claim 2, wherein for overlapping signals that are associated with the target-specific barcode sequence, each overlapping signal can be associated with the signal of step (c1) or the signal of step (c2) but not both, thereby resolving the overlapping signals into signals associated with the first and second probes, respectively.

4. The method of claim 1, wherein the plurality of signals in step (b) are detected at multiple locations in the biological sample, the signal in (c1) is detected at a first subset of the multiple locations, the signal in (c2) is detected at a second subset of the multiple locations, and wherein the first and second subsets of the multiple locations do not completely overlap.

5. The method of claim 1, wherein the signals in steps (b), (c1), and/or (c2) are detected using detectable probes that directly or indirectly bind to the target-specific barcode sequence or a complement thereof, the first probe-resolution barcode sequence or a complement thereof, and the second probe-resolution barcode sequence or a complement thereof, respectively.

6-23. (canceled)

24. The method of claim 1, wherein the first and second probe-resolution barcode sequences are different among probes targeting the same target analyte.

25. The method of claim 1, wherein the first and/or second probe-resolution barcode sequences are common among two or more probes each targeting a different target analyte in the biological sample.

26-27. (canceled)

28. The method of claim 1, wherein the first molecule of the target analyte is of a first species and the second molecule of the target analyte is of a second species different from the first species, and wherein the first and second probe-resolution barcode sequences are associated with the first and second species, respectively.

29-42. (canceled)

43. The method of claim 1, wherein the first and second probes are circularizable probes, and ends of the circularizable probes are ligated using the nucleic acid sequence in the target analyte as a template, with or without gap filling prior to ligation.

44-57. (canceled)

58. The method of claim 1, comprising:
(i) contacting the biological sample with detectable probes that hybridize to the target-specific barcode sequence or complement thereof;
(ii) imaging the biological sample to detect the plurality of signals of step (b);
(iii) optionally removing the detectable probes from the target-specific barcode sequence or complement thereof;
(iv) contacting the biological sample with detectable probes that hybridize to the first and second probe-resolution barcode sequences or complements thereof;
(v) imaging the biological sample to detect the signal of step (c1) in a first detection channel;
(vi) imaging the biological sample to detect the signal of step (c2) in a second detection channel that is different from the first detection channel; and
(vii) optionally removing the detectable probes from the first and second probe-resolution barcode sequences or complements thereof.

59. The method of claim 58, wherein the detectable probes in step (i) comprise intermediate probes that hybridize to the target-specific barcode sequence or complement thereof and detectably-labeled probes that hybridize to the intermediate probes.

60. The method of claim 58, wherein the detectable probes in step (iv) comprise intermediate probes that hybridize to the first and second probe-resolution barcode sequences or complements thereof and detectably-labeled probes that hybridize to the intermediate probes.

61. The method of claim 58, wherein:
the detectable probes in step (i) are directly or indirectly labeled with a fluorescent label that is different from fluorescent labels of the detectable probes in step (iv);
the method does not comprise step (iii);
steps of (i) and (iv) are performed simultaneously by contacting the biological sample with: detectable probes that hybridize to the target-specific barcode sequence or complement thereof, and detectable probes that hybridize to the first and second probe-resolution barcode sequences or complements thereof; and
steps (ii), (v), and (vi) are performed in any order.

62. (canceled)

63. The method of claim 58, wherein:
the detectable probes in step (i) are directly or indirectly labeled with a fluorescent label that is detectable in the same fluorescent channel as a fluorescent label of the detectable probes in step (iv);
the method comprises step (iii); and
steps (v) and (vi) are performed in any order.

64-66. (canceled)

67. The method of claim 58, further comprising repeating any one or more of steps (i)-(vii) one or more times, each time with a different plurality of detectable probes that hybridize to the target-specific barcode sequence or complement thereof, and/or with the same or a different plurality of detectable probes that hybridize to the first and second probe-resolution barcode sequences or complements thereof.

68-69. (canceled)

70. The method of claim 58, further comprising registering images of the imaging steps for detecting the plurality of signals of step (b), the signal of step (c1), and the signal of step (c2), and the plurality of signals of step (b), the signal of step (c1), and the signal of step (c2) are associated using the registered images.

71-73. (canceled)

74. A method for analyzing a biological sample, comprising:
(a) contacting the biological sample with a plurality of circular or circularizable probes comprising a first circular or circularizable probe and a second circular or circularizable probe,
wherein the first circular or circularizable probe comprises a target-specific barcode sequence and a first probe-resolution barcode sequence, and the second circular or circularizable probe comprises the target-specific barcode sequence and a second probe-resolution barcode sequence, and
wherein the plurality of circular or circularizable probes hybridize to different nucleic acid molecules in the biological sample, and the target-specific barcode sequence corresponds to a target nucleic acid;
(b) generating rolling circle amplification (RCA) products of the first and second circular or circularizable probes;
(c) contacting the biological sample with detectable probes that hybridize to the RCA products at the complement of the target-specific barcode sequence;
(d) detecting signals associated with the target-specific barcode sequence;
(e) contacting the biological sample with detectable probes that hybridize to the RCA products at the complement of the first probe-resolution barcode sequence and with detectable probes that hybridize to the RCA products at the complement of the second probe-resolution barcode sequence; and
(f) detecting, in separate detection channels, a signal associated with the first probe-resolution barcode sequence and a signal associated with the second probe-resolution barcode sequence.

75-79. (canceled)

80. The method of claim 74, wherein the signals associated with the target-specific barcode sequence in step (d) comprise overlapping signals that are not spatially resolved into individual puncta.

81-83. (canceled)

84. A method for analyzing a biological sample, comprising:
(a) contacting the biological sample with a plurality of probes each comprising a target-specific barcode sequence associated with a target analyte,
wherein a first probe of the plurality of probes comprises a first probe-resolution barcode sequence associated with a first species of organism and a second probe of the plurality of probes comprises a second probe-resolution barcode sequence associated with a second species of organism, and
wherein the first probe targets a first nucleic acid sequence of the target analyte of the first species of organism and the second probe targets a second nucleic acid sequence of the target analyte of the second species of organism, and the target-specific barcode sequence corresponds to the target analyte;
(b) detecting a plurality of signals associated with the target-specific barcode sequences of the plurality of probes;
(c1) detecting a signal associated with the first probe-resolution barcode sequence; and
(c2) detecting a signal associated with the second probe-resolution barcode sequence,
wherein the signals of steps (c1) and (c2) are associated with the target analyte.

85. The method of claim 84, wherein the first nucleic acid sequence and the second nucleic acid sequence are homologs of the target analyte in the first and second species of organism, respectively.

86-97. (canceled)
